(19) United States
(12) Patent Application Publication
(10) Pub. No.: US 2021/0253705 A1
(43) Pub. Date: Aug. 19, 2021

(54) METHODS AND AGENTS FOR TREATING ORGAN INJURY AND TRANSPLANT REJECTION

(71) Applicant: The Regents of the University of California, Oakland, CA (US)

(72) Inventor: John A. BELPERIO, Los Angeles, CA (US)

(73) Assignee: The Regents of the University of California, Oakland, CA (US)

(21) Appl. No.: 17/177,890

(22) Filed: Feb. 17, 2021

Related U.S. Application Data
(60) Provisional application No. 62/978,152, filed on Feb. 18, 2020.

(51) Int. Cl. C07K 16/28; C07K 16/42; A61K 45/06

(52) U.S. Cl. CPC C07K 16/2818; A61K 45/06; C07K 16/2866; C07K 16/4291

(57) ABSTRACT

A method is described for treating or preventing organ injury, delayed type hypersensitivity, graft-versus-host disease or allograft rejection, for example in the lung, kidney, heart, liver in a subject by administering to the subject an effective amount of a CCR4 inhibitor or antagonist.

### CROSS-REFERENCE TO RELATED APPLICATION

This application claims the benefit of U.S. Provisional Patent Application No. 62/978,152, filed Feb. 18, 2020, which is incorporated herein by reference in its entirety.

### GOVERNMENT SUPPORT

This invention was made with government support under Grant Number HL112990, awarded by the National Institutes of Health. The government has certain rights in the invention.

### BACKGROUND

Lung transplants have among the worst overall long-term clinical outcomes with a 5-year survival of less than 55%. This is particularly concerning when compared to other solid organ transplants such as liver, kidney and heart that have 5-year survival rates of at least 70%. Lung transplant recipients also have much higher rates of rejection, the main risk factor for limited lung allograft survival. Rejection is classically considered a consequence of an immune response to donor alloantigens that results in allograft dysfunction. However, many of the molecular factors involved in the initiation of the alloresponse that leads to rejection remain largely unknown.

Rejection of solid organs involves alloresponsive lymphocytes and delayed type hypersensitivity (DTH). The alloresponse begins with the homing of recipient-derived naïve T cells (Tn) to secondary lymphoid tissues (SLT). Once inside SLT, Tn traffic intranodally to antigen presenting cells (APC) and this interaction generates allo-specific T cells in a process called allopriming. The allo-specific lymphocytes egress from the SLT, traffic to the transplanted organ and release cytotoxic mediators that directly injure the allograft. The alloreactive lymphocytes also initiate a DTH response that directs waves of activated leukocytes into the allograft further destroying the injured graft, ultimately leading to graft failure.

Similarly, acute lung injury, interstitial lung diseases, COPD and acute respiratory distress syndromes all share a common theme: a leukocyte cellular infiltration to the different compartments of the lung that is involved with epithelial cell injury, endothelial cellular injury as well as fibroblast proliferation, invasion and transformation that ultimately causes lung damage.

Acute injury to other organs as well as rejection share similar pathophysiological mechanisms. It is towards reducing lung and other organ injuries that the present invention is directed.

### SUMMARY

In one aspect, a method is provided for treating or preventing organ injury, delayed type hypersensitivity, graft-versus-host disease or allograft rejection in a subject comprising administering to the subject an effective amount of a C—C chemokine receptor inhibitor or antagonist.

In one embodiment, the organ injury is lung injury. In one embodiment, lung injury is selected from acute lung injury, pulmonary fibrosis, interstitial lung diseases, COPD, acute respiratory distress syndromes and sequelae of allogeneic lung transplantation.

In one embodiment, the organ injury is kidney, heart or liver injury. In one embodiment the allograft rejection is of a kidney, heart, liver, lung, intestine, extremity, face or pancreas.

In one embodiment, the C—C chemokine receptor is CCR4. In one embodiment, the CCR4 inhibitor or antagonist is a small molecule or an antibody. In one embodiment, the antibody is mogalizumab, mogalizumab-kpkc, or KH-4F5. In one embodiment, the small molecule is CCX6239, FLX475, BMS-397, AZD-2098, AZD-1678, GSK2239633, 1′-{4-[(4-chlorophenyl)amino]-6,7-dimethoxyquinazolin-2-yl}-1,4′-bipiperidin-3-yl)methanol, 4-[4-chloro-6-(2,4-dichloro-benzylamino)-pyrimidin-2-yl]-piperazin-1-yl}-piperidin-2-yl-methanone, C-021 dihydrochloride or RPT193.

In one embodiment, the method further comprises administering to the subject a CTLA4 inhibitor. In one embodiment, the CTLA4 inhibitor is abatacept or ipilimumab.

In one embodiment, the method further comprises administering induction therapies that includes but is not limited to ATG, an IL-2R antagonist, cyclophosphamide, anti-integrin therapy, anti-CD52, or a mTOR inhibitor.

In one embodiment, the method further comprises administering to the subject one or more compounds selected from the group consisting of a maintenance immune drug, cyclosporin, tacrolimus (FK506), sirolimus (rapamycin), a mTOR inhibitor, methotrexate, mycophenolic acid (mycophenolate mofetil), everolimus, daphnoretin, dexamethasone, prednisone, azathioprine, fluorouracil, mercaptopurine, anthracycline, bleomycin, dactinomycin, mithramycin, mitomycin, NOX-100 or a biologic. In one embodiment, a biologic includes but is not limited to those modulating anti-IL-6 pathways, anti-TNF, and rituximab.

In one embodiment, the CCR4 inhibitor or antagonist is administered in an amount comprising from about 0.5 to about 50 mg/kg/day. In one embodiment, the CCR4 inhibitor or antagonist is administered in an amount comprising from about 1 to about 5 mg/kg/day. In one embodiment, the CCR4 inhibitor or antagonist is administered orally to the subject. In one embodiment, the CCR4 inhibitor or antagonist is administered parenterally to the subject. In one embodiment, the CCR4 inhibitor or antagonist is administered for between about 1 to about 180 days. In one embodiment, the CCR4 inhibitor or antagonist is administered for an indefinite period of time to maintain inhibition of transplant rejection.

In one embodiment, the CCR4 inhibitor or antagonist is administered to the subject prior to a tissue or organ transplant. In one embodiment, the CCR4 inhibitor or antagonist is administered to the donor prior to transplantation of the tissue or organ into the subject. In one embodiment, the CCR4 inhibitor or antagonist is exposed to the tissue or organ ex vivo prior to transplantation into the subject, to lung injury patients or for the prevention of lung injuries.

In one aspect, a method is provided for treating or preventing allograft rejection in a subject comprising administering to the subject an effective amount of a C—C chemokine receptor antagonist. In one embodiment, the C—C chemokine receptor is CCR4.

In one embodiment, the method further comprises administering to the subject a CTLA4 inhibitor. In one embodiment, the CTLA4 inhibitor is abatacept or ipilimumab.

### BRIEF DESCRIPTION OF FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1C. Draining allograft recipient lymph nodes have increased expression of CCL17 and CCL22.

FIGS. 2A-2L. CCR4−/− recipients of airway allografts attenuate rejection and have a reduction of T cells in their draining lymph nodes.

FIGS. 3A-3C. CCR4−/− recipients, but not CCR4−/− donors attenuate the development of murine airway allograft rejection.

FIGS. 4A-4C. CCR4−/− allograft recipients have reduced T cells in draining lymph nodes at day 7.

FIGS. 5A-5H. CCR4 expression is involved in naïve T cell (Tn) homing and intranodal activation.

FIGS. 6A-6B. CCR4 expression on T cells is important for homing to draining lymph nodes as well as intranodal T cell activation.

FIGS. 7A-7C. CCR4−/− allograft recipients have a reduction in the clonal expansion of CD4⁺ and CD8⁺ T cells.

FIGS. 8A-8H. CCR4−/− recipients have a reduction in airway allograft infiltrating cytotoxic lymphocytes and their mediators.

FIGS. 9A-9C. Cd1d−/− recipients as well as the adoptive transfer of CCR4+/+ CD8⁺ T cells to CCR4−/− recipients does not affect airway rejection.

FIGS. 10A-10E. The hierarchy for T cells re-establishing rejection in the CCR4−/− recipient mice.

FIGS. 11A-11D. CTLA4-Ig combined with CCR4−/− recipients leads to long-term allograft accommodation in two models of allograft rejection.

FIG. 12. CTLA4-Ig combined with CCR4−/− recipients leads to long-term airway allograft accommodation.

### DETAILED DESCRIPTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

Despite current immunosuppressive strategies, long-term lung transplant outcomes remain poor due to rapid allogenic responses. Using a stringent mouse model of allo-airway transplantation, the CCR4-ligand axis was identified as a central node driving secondary lymphoid tissue homing and activation of the allogeneic T cells that prevent long-term allograft survival. CCR4 deficiency on transplant recipient T cells was shown to diminish allograft injury and when combined with CTLA4-Ig led to an unprecedented long-term lung allograft accommodation. Thus, we identify CCR4-ligand interactions as a central mechanism driving allogeneic transplant rejection and thus a target to enhance long-term lung transplant survival. These observations are also applicable to allograft survival of other organs, as well as acute lung injury, pulmonary fibrosis and acute and chronic injuries of other organs.

Median survival for solid organ transplants such as liver, kidney and hearts is greater than 10 years; regrettably this falls to less than 5 years for lung transplant recipients. The “Achilles' heel” for lung transplant is chronic lung allograft dysfunction, which is predominately due to rejection. Unfortunately, current immunosuppressive strategies have been ineffective for long-term lung allograft accommodation, which mandates the need for new insights into lung alloreactivity. As noted above, we demonstrated that the CCR4-ligand biological axis is central to the allopriming that leads to graft rejection and that CCR4 deficiency dramatically abolishes rejection by restricting nodal homing and subsequent intranodal activation of CD4⁺ and CD8⁺ Tn cells. Both CCR4 expressing CD4 and CD8 T cells are critical for rejection, but the CD4⁺ T cells were pivotal for helping generate alloresponsive cytotoxic T cells that trigger rapid rejection.

As will be seen in the examples herein, CCR4 deficiency combined with a low dose CTLA4-Ig immunotherapy at the time of airway transplantation enabled an unprecedented long-term allograft survival to over 125 days, over 3 times longer than CTLA4-Ig alone. This was corroborated using the orthotopic lung transplant model. Thus, this study demonstrates a molecular mechanism involving one specific chemokine axis required for homing and intranodal activation of allo-specific CD4⁺ and CD8⁺ Tn cells that mediate graft rejection. Importantly, inhibition of this chemokine axis with CTLA4-Ig allows for long-term allograft survival.

Secondary lymphoid tissues (SLT) are strategically positioned at the interface between the blood and the lymphatic system and provides an environment that allows for the exchange of antigenic information between cells of the innate and adaptive immune system. However, the crowding of immune cells in the SLT suggests a role for chemokine guidance in optimizing adaptive immunity. Many studies suggest chemokine redundancy limits the usefulness of targeting a single chemokine axis to significantly impair T cell priming. However, the marked elevations of the CCR4 ligands from the allograft SLT led to the finding that these chemokines could be key regulators of allopriming.

Importantly, CCR4−/− allograft recipients were found to have had an unprecedented prolonged reduction in rejection when compared to other chemokine axes. This showed that inhibition of allopriming results in a superior transplant outcome as contrasted to trying to limit the consequences of allo-specific T cells that were already generated. Mechanistically, any significant effect of CCR4 expression on donor cells in direct rejection were ruled out, which focused attention on recipient CCR4 expressing T cells during allopriming.

Post-transplant, draining nodes express CCL17 localized to HEV and CCL22 from subcapsular sinus mononuclear phagocytes, which is consistent with adoptive transfer studies demonstrating that CCR4 expression from Tn (CD4⁺ and CD8⁺) was fundamental for proper homing during allopriming. Furthermore, the expression of CCL22 from paracortical mononuclear phagocyte in close proximity to HEV, in combination with data indicating that the few CCR4−/− CD4⁺ and CD8⁺ Tn cells that make it the SLT have trouble shedding CD62L, is consistent with a reduction in CCR4−/− Tn cell interactions with APC surrounding HEV. Collectively, these studies corroborate that the CCR4-ligand biological axis is unique in its importance for both CD4⁺ and CD8⁺ Tn cells to home to SLT, and for intranodal activation needed for the optimal generation of alloresponsive T cells.

The findings described here are consistent with the importance of CCR4 expression on other lymphocytes such as NKT cells, as was shown during ova sensitization. More specifically, CCR4 ligands were expressed by APC within SLT, which optimized their interactions with NKT cells; that in turn, licensed the APC to express CCL3 and CCL4 calling in CCR5⁺CD8⁺ T cells, ultimately generating ova specific cytotoxic T lymphocytes. Thus the role of NKT cells was explored during allograft rejection though the use of genetically altered mice that lack NKT cells (i.e., Cd1d1−/− mice) as allograft recipients. However, no difference were found in early or late allograft rejection, essentially negating any substantial role of CCR4 expressing NKT cells during rejection.

CCR4−/− allograft recipients were noted to have a greater reduction in the frequency of CD8⁺ T cells as compared to CD4⁺ T cells in both the allografts and SLT, which led to the evaluation if whether CCR4 expression on CD8⁺ T cells could lead to CD8⁺ T cell help in generating more allo-injurious T cells. However, adoptive transfer studies did not establish any CCR4+/+CD8⁺ T cell help with regard to rejection. Conversely, CCR4 expression on CD4⁺ T cells was not found to be important for CD4⁺ T cell help in generating an alloreactive response causing rejection. Taken together, these adoptive transfer studies demonstrate a hierarchy of CCR4+/+ lymphocytes with respect to re-establishing rejection in the CCR4−/− recipients (CCR4+/+CD90.2>CCR4+/+CD4⁺ T cells>CCR4+/+CD8⁺ T cells). Importantly, perturbing allopriming via CCR4-ligand interruption had physiologic consequences such as a marked reduction in the clonal expansion of both CD4⁺ and CD8⁺ alloresponsive T cells, decreased in vivo DTH response to alloantigens, and diminished cytotoxic T cell down-stream pathways at the site of the allograft. The studies described here suggest CCR4-ligand interaction is a linchpin in the alloresponse, and inhibition of this single chemokine axis can limit allograft rejection.

Rodent models of allograft rejection have demonstrated that the most impressive therapy that allows for long-term allograft acceptance was the combined costimulatory blockade involving anti-CD154 and CTLA4-Ig. Unfortunately, human studies involving anti-CD154 led to high rates of vessel thrombosis quickly dampening the hopes for a strategy to achieve long-term accommodation. However, the data described here suggest that the inhibition of CCR4-ligand interactions is an alternate method to limit costimulation between CD4⁺ Tn cells and APC. Thus, we tested the combination of a short course of CTLA4-Ig with either the CCR4−/− or CCR4+/+ recipients and found that CCR4−/− recipients have virtually normal airway allografts at day 126, while CCR4+/+ receipts were completely rejected by day 42. Importantly, this was reproducible in the vascularized orthotropic single lung transplant model as CTLA4-Ig given to CCR4−/− recipients rendered a virtually normal lung allograft at day 126.

Thus, it was found that interrupting allopriming by manipulating CCR4-ligand interactions causes both CD4⁺ and CD8⁺ Tn cells to have difficulties homing to SLT, as well as their activation within the node. This leads to a dramatic reduction in the clonal expansion of alloresponsive T cells, DTH response, and cytotoxic mediators, which markedly attenuates allograft rejection. Moreover, the combination of CCR4-ligand inhibition with CTLA4-Ig blockade leads to long-term accommodation that outperforms the best known combination of costimulatory blockade (anti-CD154 and CTLA4-Ig). Overall, the studies herein suggest that altering events prior to allorecognition, or the so called “Signal 0”, via the CCR4-ligand biological axis may be a therapeutic option to prolong allograft survival and warrants further investigation in human organ transplantation.

In one embodiment, the inhibition of CCR4 may be achieved by any number of methods. In one embodiment, an antibody or antibody fragment may be used. In one embodiment, a small molecule may be used.

Antibodies useful for the practice of the invention include, in one embodiment, mogamulizumab, also called mogamulizumab-kpkc, known as KW-0761, a humanized monoclonal antibody developed by Kyowa Hakko Kirin that was approved by the US FDA (tradename POTELIGEO).

In one embodiment, the antibody is KH-4F5.

In one embodiment, the CCR4 inhibitor or antagonist is a fragment of any of the foregoing antibodies.

In one embodiment, the small molecule CCR4 inhibitor or antagonist is CCX6239.

In one embodiment, the small molecule CCR4 inhibitor or antagonist is FLX475

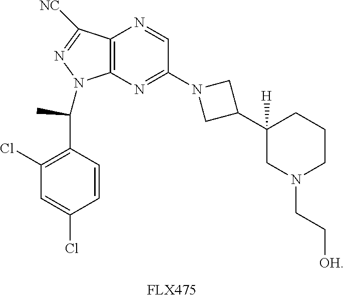 [SMILES: C[C@H](c1ccc(Cl)cc1Cl)n1nc(C#N)c2ncc(N3CC([C@H]4CCCN(CCO)C4)C3)nc21]

In one embodiment, the small molecule CCR4 inhibitor or antagonist is BMS-397 ((R)-(4-(4-((2,4-dichlorobenzyl)amino)pyrido[2,3-d]pyrimidin-2-yl)piperazin-1-yl)-piperidin-2-yl-methanone).

In one embodiment, the small molecule CCR4 inhibitor or antagonist is AZD-2098 (2,3-dichloro-N-(3-methoxypyrazin-2-yl)benzenesulfonamide).

In one embodiment, the small molecule CCR4 inhibitor or antagonist is AZD-1678 (2,3-dichloro-N-(5-fluoro-3-methoxypyrazin-2-yl)benzenesulfonamide).

In one embodiment, the small molecule CCR4 inhibitor or antagonist is GSK2239633 (N-(3-((3-(5-chlorothiophene-2-sulfonamido)-4-methoxy-1H-indazol-1-yl)methyl)benzyl)-2-hydroxy-2-methylpropanamide).

In one embodiment, the small molecule CCR4 inhibitor or antagonist is 1′-{4-[(4-chlorophenyl)amino]-6,7-dimethoxyquinazolin-2-yl}-1,4′-bipiperidin-3-yl)methanol.

In one embodiment, the small molecule CCR4 inhibitor or antagonist is 4-[4-chloro-6-(2,4-dichloro-benzylamino)-pyrimidin-2-yl]-piperazin-1-yl}-piperidin-2-yl-methanone.

In one embodiment, the small molecule CCR4 inhibitor or antagonist is 2-[1,4′-bipiperidin]-1′-yl-N-cycloheptyl-6,7-dimethoxy-4-quinazolinamine dihydrochloride, also known as C-021 dihydrochloride.

The CCR4 inhibitor or antagonist may be administered to a subject at a dose level, dosing frequency, and duration to achieve the desired effect. The CCR4 inhibitor or antagonist may be administered by a route consistent with optimally delivering its desired effects. An antibody may be administered parenterally, such as but not limited to intravenously or subcutaneously. A small molecule may be administered orally or parenterally. In one example, the CCR4 inhibitor or antagonist is administered in an amount comprising from about 0.5 to about 50 mg/kg/day. In one example, the CCR4 inhibitor or antagonist is administered in an amount comprising from about 1 to about 5 mg/kg/day.

The CCR4 inhibitor or antagonist may be administered orally to the subject. The CCR4 inhibitor or antagonist may be administered parenterally to the subject.

The CCR4 inhibitor or antagonist may be administered for between about 1 to about 180 days. The CCR4 inhibitor or antagonist may administered for an indefinite period of time to maintain inhibition of transplant rejection.

The CCR4 inhibitor or antagonist may be or also may be administered to the subject prior to a tissue or organ transplant. The CCR4 inhibitor or antagonist may be or also may be administered to the donor prior to transplantation of the tissue or organ into the subject. The CCR4 inhibitor or antagonist may be or also may be exposed to the tissue or organ ex vivo prior to transplantation into the subject. Any dosing or exposure regimen, ex vivo, in vivo, in the donor, or any other modality may be carried out in order to optimally prevent or reduce disease.

In one embodiment, the method further comprises administering to the subject a CTLA4 inhibitor. In one embodiment, the CTLA4 inhibitor is abatacept, which is a fusion protein composed of the Fc region of the immunoglobulin IgG1 fused to the extracellular domain of CTLA-4. Its tradename is ORENCIA. In one embodiment, the CTLA4 inhibitor is ipilimumab, which is a monoclonal antibody that targets CTLA-4. Its tradename is YERVOY.

The CTLA4 inhibitor may be administered to a subject at a dose level, dosing frequency, and duration to achieve the desired effect. The CTLA4 inhibitor may be administered by a route consistent with optimally delivering its desired effects. An antibody may be administered parenterally, such as but not limited to intravenously or subcutaneously. A small molecule may be administered orally or parenterally. The CTLA4 inhibitor may be administered simultaneously with, or before, or after, the CCR4 inhibitor or antagonist. The CCR4 and the CTLA4 agents may be present in the same pharmaceutical composition, or separate. Each is administered in a dosing regimen than optimizes the desired effect on the disease or rejection process. Each is delivered at the dose, frequency, duration, and timing relative to the onset of the disease, for optimal benefit of the subject or patient.

As noted above, in addition to improving the outcome of lung transplantation, other lung injuries as well as other organ injuries including those from transplantation are amenable to the teachings herein. Rejection of solid organs involves alloresponsive lymphocytes and delayed type hypersensitivity (DTH) which are addressable by the teachings herein.

Similarly, acute lung injury, interstitial lung diseases, COPD and acute respiratory distress syndromes all share a common theme: a leukocyte cellular infiltration to the different compartments of the lung that is involved with epithelial cell injury, endothelial cellular injury as well as fibroblast proliferation, invasion and transformation that ultimately causes lung damage. The methods of the invention comprising treatment with a CCR4 inhibitor or antagonist, optionally in combination with a CTLA4 inhibitor, are provided.

In one embodiment, the organ injury is kidney injury. In one embodiment, the organ injury is allograft rejection of a kidney.

In one embodiment, the organ injury is heart injury. In one embodiment, the organ injury is allograft rejection of a heart.

In one embodiment, the organ injury is liver injury. In one embodiment, the organ injury is allograft rejection of a liver.

In one embodiment, the organ injury is pancreatic injury. In one embodiment, the organ injury is allograft rejection of a pancreas.

In one embodiment the organ injury is rejection of a transplanted face. In one embodiment the organ injury is rejection of a transplanted extremity such as but no limited to a hand, foot, arm, leg or genitalia. The allograft transplantation of any other bodily part of organ from one human to another. In one embodiment, the transplantation is a xenograft transplant from a non-human species to human.

In one embodiment, the organ injury is rejection of any transplanted tissue or organs from one non-human species into another non-human species, the same or different from the donor. In one embodiment, the appropriate species-specific biologic agent as described above is used, to optimize efficacy and limit adverse effects. In other embodiment, the teachings herein on organ injury are equally applicable to those in non-human species, including but not limited to domestic animals, pets and livestock.

The following examples are presented in order to more fully illustrate the preferred embodiments of the invention. It should in no way be construed, however, as limiting the broad scope of the invention.

### EXAMPLES

### Example 1

### Materials and Methods

Murine Rejection Models. Heterotopic subcutaneous tracheal/airway transplantation model of alloreactivity involves the most stringent MHC class I- and II-disparate combination mismatch found in mice: BALB/c (H-2^{d}) tracheas transplanted subcutaneously into the upper backs of C57BL/6 (H-2^{b}) mice (allografts), and C57BL/6 tracheas transplanted subcutaneously into the backs of C57BL/6 mice (syngeneic control). Each mouse was subcutaneously transplanted with two tracheas. Small molecule CCR4 inhibitor or antagonist C 021 dihydrochloride (Cat #3581, Tocris Bio-Techne, Minneapolis, Minn., USA) was resuspended in sterile DMSO to 100 mg/ml and diluted further in 1×PBS to be administered as intraperitoneal (i.p.) injections at 50 mg/kg daily starting at day −1 prior to tracheal transplantation until day 14 when allografts were harvested for rejection analysis. For long-term experiments, CTLA4-Ig treatment (Bristol-Myers Squibb Company, Princeton, N.J., USA) was given as 0.2 mg i.p. injection on day 0 prior to tracheal transplantation and at days 2, 4 and 6 thereafter.

The orthotopic left single lung transplant model involved BALB/c left lungs transplanted into CCR4+/+ or CCR4−/− recipients in combinations with a single pre-operative dose of 0.1 mg CTLA4-Ig using a modification of the rodent left lung transplant. Donor BALB/c mice were anesthetized, intubated, ventilated and the left lung harvested and Teflon cuffs secured on the pulmonary artery, vein and bronchus then covered with soaked gauze pads on ice. The recipient mice are anesthetized, intubated, and ventilated. A 2-cm incision is made between 3-4 ribs on the left and a ¼-binder clip placed to expose the posterior hilum. Appropriate dissection of the hilum components included ligation and clamping after heparin delivery. A donor lung is inserted using the cuff technique via 10.0 ethilon suture. Incisions closure involved v6-0 surgical suture. During the procedure 1 cc of saline is administered subcutaneously and post-operatively the animals are placed in cages on warm water mattresses until recovered from anesthesia. Buprenorphine 0.1 mg/kg i.p. is given every 12 hours while monitoring for pain. Sutures are removed on post-operative day 7.

Histopathological Evaluation of Airway and Lung Transplants. Airway and lung graft tissues were fixed overnight in 4% paraformaldehyde, processed, paraffin-embedded, sectioned and stained with hematoxylin and eosin (H&E) or Elastin Trichrome. Three independent blinded reviewers calculated the degree of airway injury using a histological scoring system. All qualitative histological changes were evaluated and the total murine rejection score was calculated based on four pathological criteria: (a) airway lining epithelial loss, (b) deposition of extracellular matrix (ECM), (c) leukocyte infiltration, and (d) luminal fibro-obliteration due to granulation tissue formation and/or fibrosis. Each process was scored on a scale of 0-4 (0=normal, 1=mild, 2=moderate, 3=severe and 4=very severe) and added together for a total rejection score. Lung allograft rejection scores included acute rejection, lymphocytic bronchiolitis, pleural and septal rejection based on established criteria. Images were taken by Zeiss Axioskop 2 plus microscope and analyzed with AxioVision 4.2 imaging software. Images were taken as 5× to show whole pathology of tracheas and as 20× to amplify presence or absence of either epithelial cells and/or fibro-obliteration of the airway allograft lumen as well as different aspects of lung rejection and fibrosis.

Immunohistochemistry. Staining of paraffin-embedded lymph node samples was performed using the VECTASTAIN ABC Standard kits. After deparaffinization and antigen retrieval in sodium citrate buffer (pH 6.0), endogenous perioxidase was quenched with 3% hydrogen peroxide. Tissues were incubated in appropriate blocking serum and endogenous biotin was blocked with an avidin/biotin blocking kit. Slides were incubated overnight with primary antibodies at 4° C. The primary antibodies for rat polyclonal anti-mouse CCL17 was MAB529 and rabbit monoclonal anti-mouse CCL22 was ab124768. Specific labeling was detected with a biotinylated specific secondary antibody and application of horseradish peroxidase-conjugated avidin-biotin followed by visualization with DAB solution.

Total RNA isolation and real-time quantitative PCR. Total cellular RNA from transplanted airways (previously frozen in liquid nitrogen) was isolated using Trizol and chloroform treatment, precipitated with isopropanol, washed twice with 75% ethanol and resuspended in DEPC-treated water. Total RNA concentration was determined using Nanodrop 2000 UV-Vis spectrophotometer and 2 μg of total RNA was DNase-treated to remove genomic DNA contamination and reversed transcribed into cDNA using Taqman reverse transcription reagents. Specific targets were amplified on StepOnePlus qPCR machine using Taqman gene expression assays: Perforin 1 (Mm00812512_m1), Fas-ligand (Mm00438864_m1), Foxp-3 (Mm00475156_m1) and Eukaryotic 18S (4319413E) as an endogenous control. The RNA expression levels were compared to wild-type allografts using 2^{ΔΔCt} method and visualized as fold difference to allografts.

Protein Analysis by Luminex. Draining lymph nodes and heterotopically transplanted airways from recipient animals were surgically removed on different days (7, 14 and 21 days) and snap frozen in liquid nitrogen. Frozen tissues were homogenized in 1×PBS supplemented with complete protease inhibitor cocktail, sonicated on ice for 10 seconds, centrifuged at 13,000 rpm to remove any cellular debris and analyzed by Luminex labeling kits using manufacturer's instructions.

Flow Cytometry Analysis of draining lymph nodes and airway allografts. Single-cell suspension was prepared from harvested axillary/brachial lymph nodes or airways. Briefly, tissues were put through a steel mesh by using a plunger and cells were collected in RPMI-1640 media supplemented with L-glutamine, penicillin/streptomycin and HEPES buffer. Red blood cells were lysed using ACK (Ammonium-Chloride-Potassium) lysing buffer and washed twice in 1×PBS+0.1% FBS. Live cells counted with hemocytometer using trypan blue and 1×10⁶ cells were stained with different cell surface conjugated anti-mouse antibodies: hamster CD3a-PerCP, CD3a-FITC, rat CD4-FITC, CD4-APC, CD8-FITC, CD8a-APC, CD44-APC, and CD62L-PE. The cell suspensions were acquired using FACSCalibur, analyzed with CellQuest software and data was expressed and compared as frequencies of selected subpopulations. The specific gating strategies for each experiment are explained in other examples herein.

Flow cytometry IFN-γ cytokine secretion assay (FCIS). A mixture of draining lymph node (axillary/brachial) cells from CCR4−/− and CCR4+/+ airway allograft recipients at day 7 were collected for ex vivo stimulation. Single cell suspension was prepared as described previously and 1×10⁶ recipient cells from CCR4−/− and CCR4+/+ allograft recipients, naïve non-transplanted CCR4−/− and CCR4+/+ mice were stimulated in the presence of either RPMI 1640 supplemented with 5% FBS alone or with 2×10⁶ irradiated and digested BALB/c splenocytes. After 16 hours of incubation at 37° C. and 5% CO2, cells were washed and analyzed using the flow cytometry IFN-γ cytokine secretion assay via flow cytometry analysis on a minimum of 250,000 total events. The cells were stained for (7-AAD (to exclude dead cells in PerCP channel), B220-PerCP (to exclude B cells), CD3-FITC, IFN-γ-PE and either CD4-APC or CD8-APC) to measure % cell frequency of alloresponsive T cells. The coefficient of variation with FCIS is 5-15% with a lower limit of detection of 0.01% or 1/10,000 IFN-γ secreting cells.

In vivo delayed-type hypersensitivity (DTH) response. Recipient animals (CCR4+/+ and CCR4−/−) post-airway transplant at day 7 were challenged with BALB/c alloantigens in a DTH response using the pinnae swelling assay. Briefly, at 7 days' post-transplant a 10 μl volume of irradiated single-cell suspension of digested BALB/c splenocytes (7.5×10⁶ total cells in saline) was injected in the right ear pinna, by using a 30-gauge needle and a Hamilton syringe. The left control ear pinna received 10 μl of sterile saline solution. Ear swelling was measured 48 hours later with a Mitutoyo 7326 Micrometer, and the results were expressed as the mean swelling of challenge ear minus the mean swelling of control ear (units, mm). All challenges and measurements were performed under light anesthesia (isoflurane).

Adoptive transfer experiments. Total (CD90.2⁺), CD4⁺ or CD8⁺ T cells were purified from spleens of naïve CCR4+/+ or CCR4−/− mice by positive selection using CD90.2, CD4(L3T4) or CD8(Ly-2) T cell Microbead Isolation Kits, respectively following manufacturing instructions. Briefly, animal spleens were surgically extracted and pushed through metal mesh to remove cells and washed twice in MACS rinsing buffer supplemented with 2% BSA. Total cells were counted using Crystal Violet and stained with MicroBeads and purified using positive selection by magnetic LS columns, washed and resuspended in sterile saline. After selection, 1×10⁷ CD90.2, 5×10⁶ of CD4⁺ or 5×10⁶ of CD8⁺ live T cells (as determined by trypan blue staining) were injected i.v. into each mouse prior to subcutaneous transplantation with BALB/c tracheas. Allograft rejection was analyzed at day 21 by histopathological rejection scoring analysis as previously described. Additionally, we performed in vivo CD8⁺ T cell depletion experiments. CCR4−/− mice were given an i.p. injection of either 0.25 mg of anti-CD8 antibody (clone 53-6.72) or control isotype antibody (clone 2A3) at day −1 of heterotopic BALB/c transplant and repeated at days 7 and 14 post-transplant with the addition of a transfer of 5×10⁶ CD4⁺ T cells injected immediately prior to heterotopic transplant procedure. Allografts were isolated at day 21 and evaluated for rejection.

T-cell trafficking. Splenic total T cells were purified from naïve CCR4+/+ or CCR4−/− animals and labeled with 4.0 μM of CFSE or 0.25 μM of CFSE, respectively, by manufacturer's instructions. Stained single cell suspensions were washed three times in cold 1×PBS/1% BSA to remove residuals of CFSE, resuspended in sterile saline and mixed as a 1:1 ratio of live cells as determined by trypan blue counts. 10×10⁶ live total T-cells per 150 μl of sterile saline were injected into (1) day 7 CCR4+/+ or CCR4−/− recipients transplanted with BALB/c airways and (2) day 7 CCR4+/+ recipients transplanted with C57BL/6 airways (isogeneic). Axillary/brachial lymph nodes were removed after 18 hours and analyzed by flow cytometry for different T cell subpopulations.

Statistics. Data were analyzed using GraphPad Prism 7.00 statistical software (GraphPad Software, La Jolla, Calif., USA, www.graphpad.com). Group comparisons were evaluated by the unpaired two-tailed t-test or Mann-Whitney where appropriate for statistical significance and reported as mean±standard error of the mean (SEM). Multiple comparisons were performed with the Kruskal-Wallis and post-hoc Dunn. p<0.05 is considered statistically significant.

### Example 2

### Draining SLT has Increased CCL17 and CCL22 Expression During Allograft Rejection

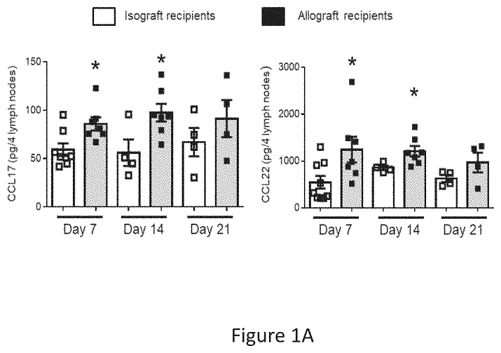

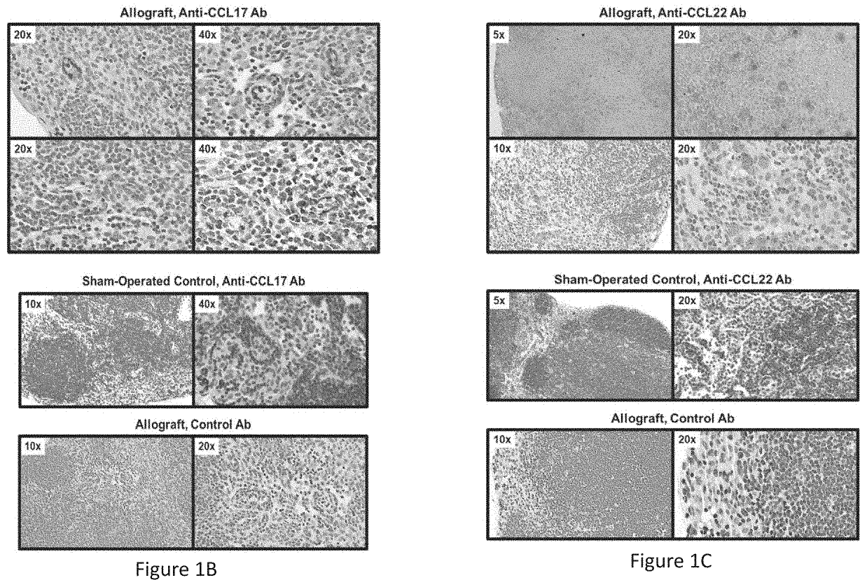

Previous studies have demonstrated that Tn cells express the CCR4 receptor while mononuclear phagocytes and APC can be a rich source for its ligands CCL17 and CCL22. We used the fully mismatched heterotopic tracheal transplant model of airway allograft rejection to explore the role of CCR4-ligands interaction in allograft rejection. BALB/c airways were transplanted subcutaneously into C57BL/6 recipients (allografts) and C57BL/6 airways into C57BL/6 recipients (isografts). This model of rejection is a highly reproducible and over time results in pathology that is representative of human acute rejection and chronic lung allograft dysfunction. A kinetic evaluation of the CCR4 ligands from whole draining SLT (axillary and brachial nodes) homogenates using Luminex technology demonstrates marked elevations of both CCL17 and CCL22 protein concentrations at days 7 and 14 from allografts, as compared to isografts (FIG. 1A). We also determined the cellular sources of these chemokines by performing immunohistochemical (IHC) analysis on allograft draining SLT (n=4) at day 7 post-transplant. Morphometrically, we observed that CCL17 is predominately expressed from high endothelial venules (HEV) in the paracortical areas (FIG. 1B). CCL22 protein localized predominately to the paracortical and subcapsular sinus mononuclear phagocytes (FIG. 1C). These chemokine expression patterns are poised to work together in bringing Tn to SLT as well as allow them to traffic within the node to APC.

As shown in FIG. 1, draining allograft recipient lymph nodes have increased expression of CCL17 and CCL22. BALB/c airways were transplanted subcutaneously into C57BL/6 recipients (allografts), as compared to C57BL/6 airways transplanted into C57BL/6 recipients (isografts). Whole allograft draining nodes were harvested at days 7, 14, and 21 for protein analysis. (1A) Bar graphs indicate the protein concentrations of CCL17 and CCL22 by Luminex from whole draining node homogenates from allograft and isograft recipients.

FIGS. 1B-1C representative immunohistochemical (IHC) staining for CCL17 and CCL22, as compared to appropriate control antibodies from allograft draining lymph nodes or sham operated CCR4+/+ mice lymph nodes at day 7. Allograft recipient CCL17 protein is expressed morphologically from HEV, as compared to virtually no staining in CCR4+/+ sham operated controls or for the control antibody. Allograft recipients CCL22 protein is detected morphologically on mononuclear phagocytes in the paracortical and subcapsular sinus, as compared to just a few mononuclear phagocytes only in the subcapsular sinus from the CCR4+/+ sham operated controls or virtually no staining for the control antibody. Protein data is representative of 4-9 mice per group. Error bars indicate SEM. Significance was determined by the Mann-Whitney test, *p<0.05. IHC experiments involve (n=4) nodes from 4 different allograft recipients.

### Example 3

The Inhibition of CCR4 Interactions with its Ligands Profoundly Attenuates Allograft Rejection

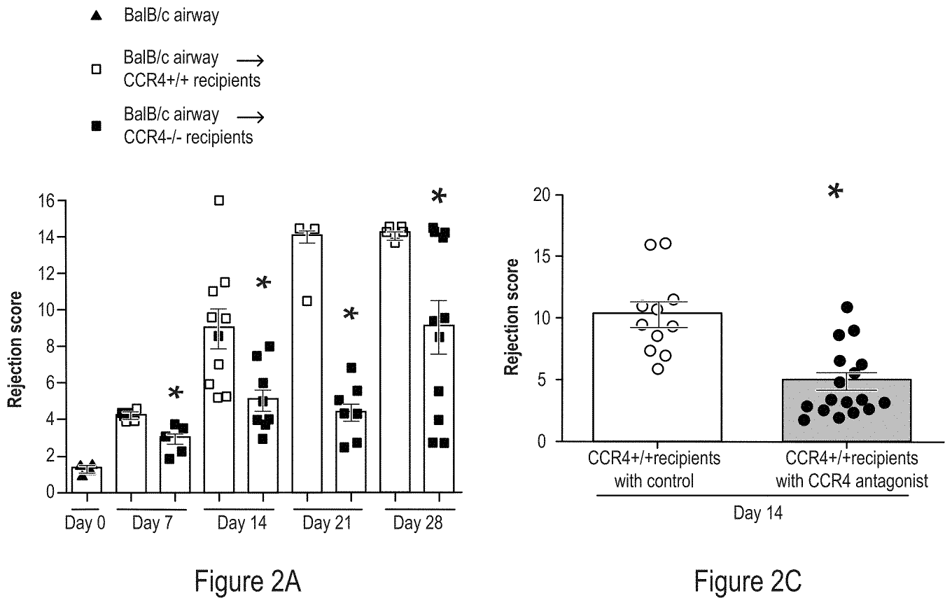

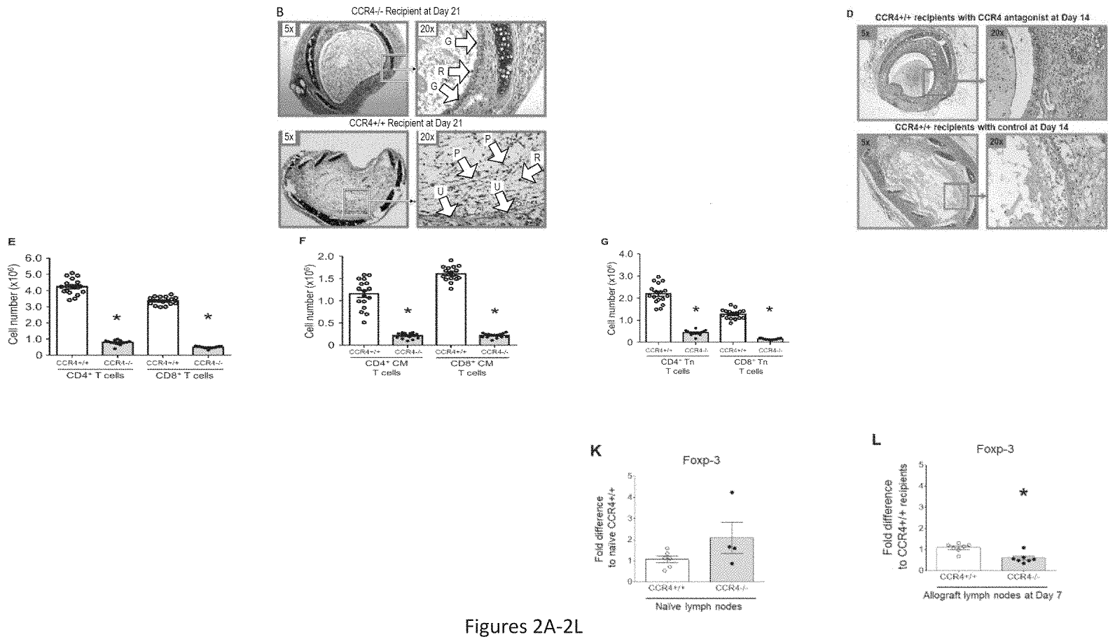

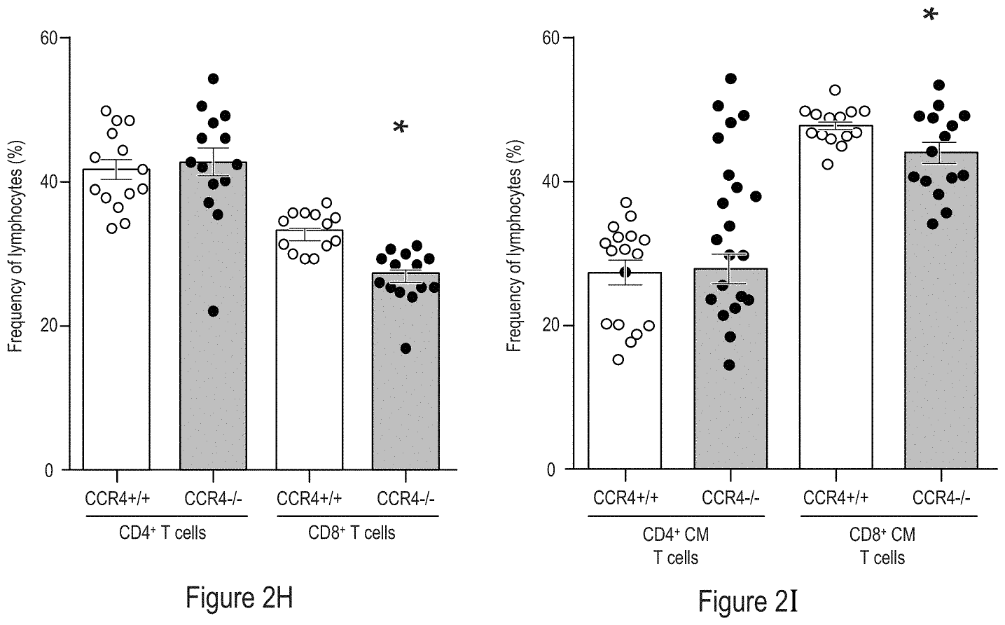

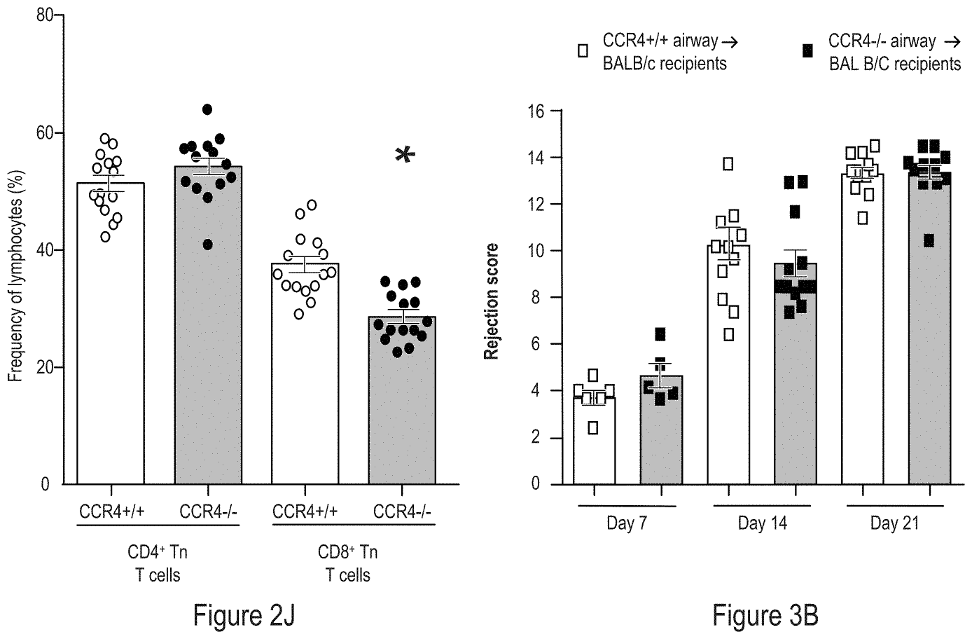

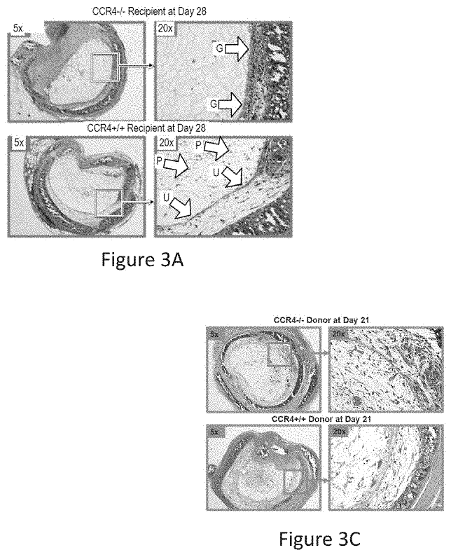

The increased levels of CCL17 and CCL22 in SLT from allograft recipients suggested that perturbing the CCR4-ligand axis could inhibit allograft injury. To test this, BALB/c airways were transplanted into C57BL/6 CCR4−/− or CCR4+/+ recipients and the allografts were harvested at multiple time points for histopathological rejection scoring based on leukocyte infiltration, epithelial injury, matrix deposition and fibro-obliteration. BALB/c donor airways prior to transplant demonstrate minimal inflammation, normal epithelium and no matrix deposition or fibrosis (FIG. 2A). CCR4−/− recipients had profound reductions in rejection, as compared to CCR4+/+ recipients at days 7, 14, 21 and 28 (FIGS. 2A and 2B, and FIG. 3A). More specifically, the allografts from the CCR4+/+ recipients developed marked leukocyte infiltration with epithelial cell injury at day 7, persistent inflammation with partially denuded epithelium and matrix deposition at day 14 and a denuded epithelium with invading fibroblasts obstructing the allograft airways at days 21 and 28. In contrast, the airway allografts from the CCR4−/− recipient mice had mild to moderate inflammation with a preserved epithelium and no significant matrix deposition or fibroblast obliteration throughout the 28-day time course (FIGS. 2A, 2B, and FIG. 3A). Interestingly, a recent study insinuates that CCR4 is required for T cell development, therefore the CCR4−/− recipient mice could have an altered T cell repertoire, which could be responsible for the reduction in allograft rejection. Thus, we performed confirmatory studies involving donor BALB/c airways transplanted into C57BL/6 recipients treated with either a CCR4 inhibitor or antagonist or an appropriate control. More specifically, recipient mice were treated with the small molecule CCR4 inhibitor or antagonist C 021 dihydrochloride (Tocris) at 50 mg/kg versus appropriate control administered intraperitoneally every day beginning at day −1 until allograft harvesting for rejection scoring at day 14. The CCR4 inhibitor or antagonist led to similar reductions in rejection scores as the CCR4−/− recipients when compared to appropriate controls (FIGS. 2C and 2D). These results suggest that the CCR4−/− recipients ability to attenuate allograft rejection is not due to altered microbiota or possibly altered CCR4−/− T cell repertoire.

CCR4 ligands have been implicated in the recruitment of APC to draining SLT. Furthermore, it has been established that donor-derived APC can migrate from the transplanted organ to the recipient SLT where they are involved in the initiation of rejection. To test the role of CCR4 on donor APC, we transplanted donor airways from C57BL/6 CCR4−/− mice into BALB/c recipients so that only the donor-derived cells would lack CCR4. However, in this situation we did not observe any alterations in allograft rejection scores at days 7, 14 or 21; and both CCR4+/+ and CCR4−/− donor airways allografts had similar amounts of infiltrating leukocytes, destroyed epithelium, and fibroblasts obstructing the airway (FIGS. 3B and 3C). Thus, CCR4 expression on host-derived cells participates in promoting allograft rejection.

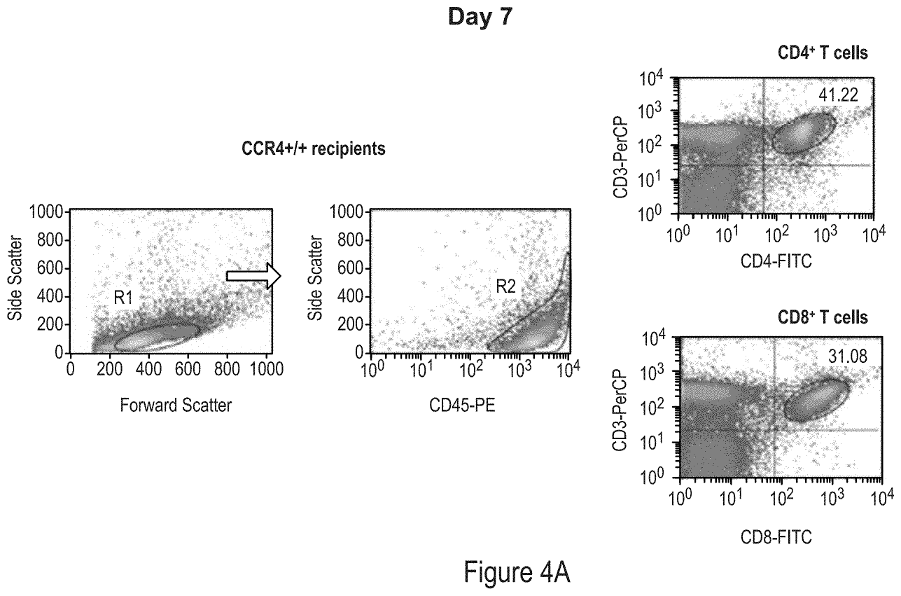

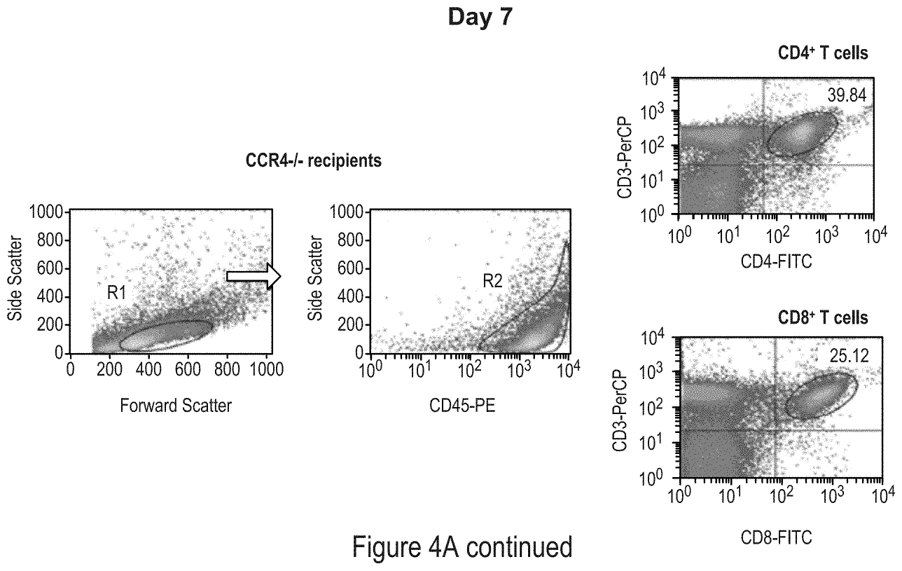

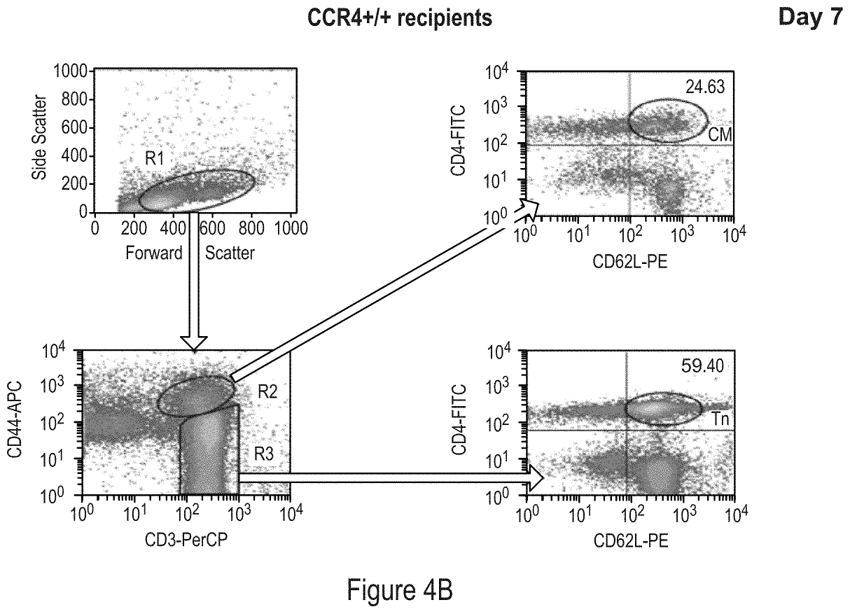

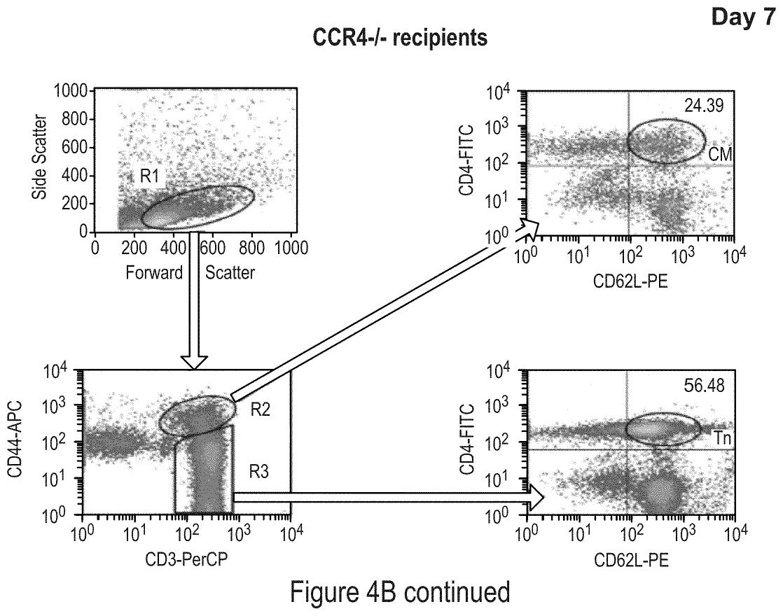

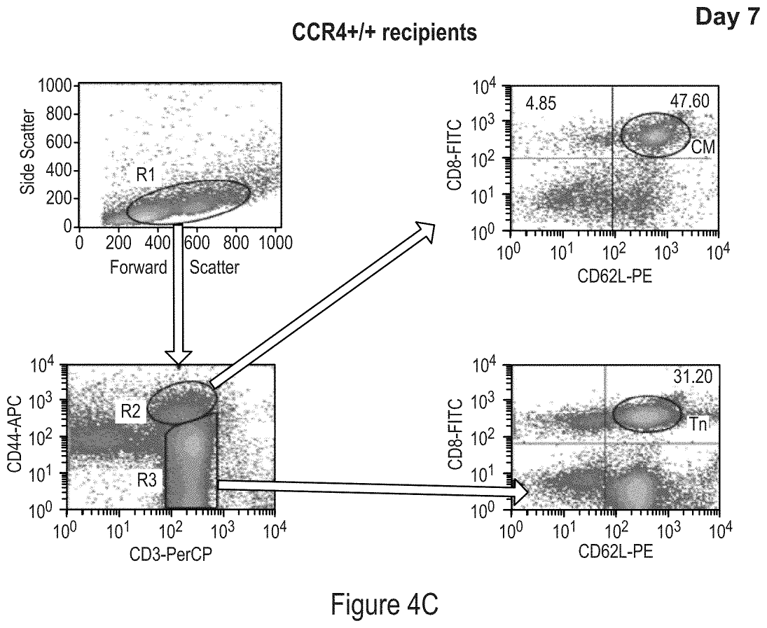

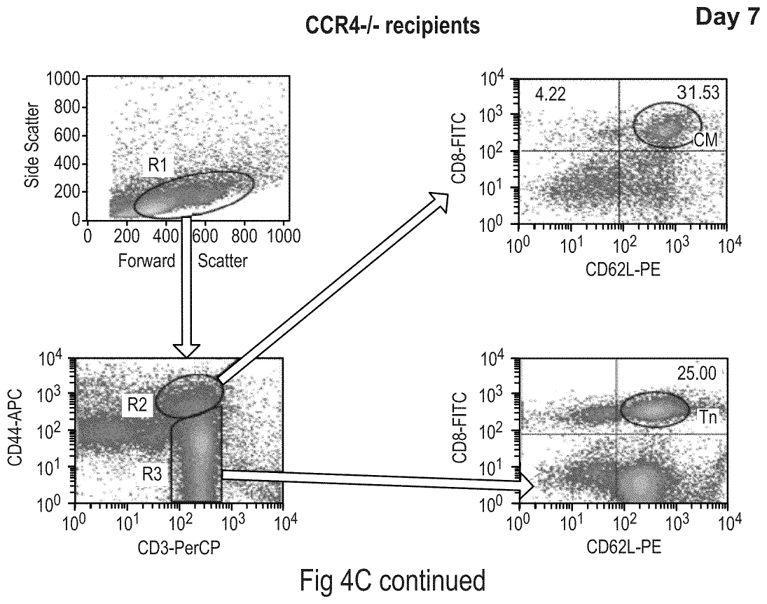

As shown in FIG. 2, CCR4−/− recipients of airway allografts attenuate rejection and have a reduction of T cells in their draining lymph nodes. BALB/c airways subcutaneously transplanted into CCR4−/− and CCR4+/+ recipients and their allografts were analyzed for rejection scores while their whole draining lymph nodes were analyzed for T cell subpopulations via flow cytometry. (A) Bar graph indicates the rejection scores for donor BALB/c airways as well as allografts from either the CCR4−/− or CCR4+/+ recipients at days 7, 14, 21 and 28. (B) Representative H&E staining of allografts from CCR4−/− and CCR4+/+ recipients at day 21. CCR4−/− recipients have limited intraluminal inflammation (R [red] arrows), virtually normal epithelium (G [green] arrows) and minimal matrix deposition without fibroblasts obstructing the lumen (i.e., fibro-obliteration). Section of the allograft is magnified to highlight the presence of a normal epithelial layer in the airway allografts from the CCR4−/− recipients. Allografts from the CCR4+/+ recipients have a moderate amount of intraluminal inflammatory cells (R [red] arrows), an absence of airway epithelial cells (U [purple] arrows) and a presence of fibroblasts (P [pink] arrows) causing fibro-obliteration of the lumen. See also FIG. 3. (C) Bar graph indicates the rejection scores of allografts from recipients treated with either the CCR4 inhibitor or antagonist or appropriate control at day 14. (D) Representative H&E staining of allografts from the CCR4 inhibitor or antagonist and control at day 14. Recipients with the CCR4 inhibitor or antagonist have a virtually normal epithelium and minimal matrix deposition without fibro-obliteration. Section of the allograft is magnified to show the presence of a normal epithelial layer in the airway allografts from the recipients treated with the CCR4 inhibitor or antagonist. Allografts from the control treated recipients have an absence of airway epithelial cells. (E-J) Bar graphs indicate the total number and frequency of CD4⁺ and CD8⁺ T cells and there naïve (Tn) and central memory (CM) T cell subpopulations from CCR4+/+ and CCR4−/− allograft recipient lymph nodes at day 7. See also FIG. 4. (K) Bar graph indicates the lymph node expression of Foxp-3 by qPCR for naïve non-transplanted CCR4+/+ and CCR4−/− mice nodes as well as (L) CCR4+/+ and CCR4−/− allograft recipient nodes at day 7. Data is representative of 4-15 mice per group. Error bars indicate SEM. Significance was determined by Mann-Whitney or unpaired t test where appropriate, *p<0.05.

As shown in FIG. 3, CCR4−/− recipients, but not CCR4−/− donors attenuate the development of murine airway allograft rejection. BALB/c airways were subcutaneously transplanted into CCR4−/− versus CCR4+/+ recipients and in separate experiments CCR4−/− versus CCR4+/+ airways were transplanted into BALB/c recipients. (A) Representative H&E staining of airway allografts from CCR4−/− and CCR4+/+ recipients at day 28. Section of the airway allograft is magnified to show the presence of a virtually normal epithelial layer (G [green] arrows) in the airway allografts from the CCR4−/− recipients. There is an absence of airway epithelial cells (U [purple] arrows) and a presence of fibroblasts (P [pink] arrows) causing fibro-obliteration of the airway allografts from the CCR4+/+ recipients. (B) Bar graph indicates rejection scores for CCR4−/− as compared to CCR4+/+ donor airways transplanted into BALB/c recipients at days 7, 14 and 21. (C) Representative H&E staining at day 21 demonstrates complete lumen fibro-obliteration from both CCR4−/− and CCR4+/+ donor airway allografts when transplanted into BALB/c recipients. Data is representative of 5-15 mice per group. Error bars indicate SEM. Significance was determined by Mann-Whitney, *p<0.05.

### Example 4

CCR4−/− SLT have a Reduction in the Amount of T Cells During Airway Allograft Rejection

Based on the HEV and mononuclear phagocytes expressing CCL17 and CCL22 in the allograft draining SLT, we tested the chemokines importance in driving T cell trafficking to the lymph nodes following allograft transplantation. At day 7 post-transplant, a time point with extensive leukocyte infiltration into the allograft, we observed a significant reduction in total numbers of CD4⁺ and CD8⁺ T cells, as well as their subpopulations with central memory (CD62L+CD44high) and naïve T cell (CD62L+CD44low/neg) phenotypes from CCR4−/− recipient lymph nodes as compared to controls (FIGS. 2E-2G). Interestingly, only the frequency of CD8⁺ T cells and their central memory and naïve T cell phenotypes were reduced, while the frequency of CD4⁺ T cells and their subpopulations of central memory and naïve T cell phenotypes was unchanged in CCR4−/− SLT compared to CCR4+/+ controls (FIGS. 2H-2J, and FIGS. 4A-4C). Some studies have shown that CCR4 expressing regulatory T cells (Treg) are important for tolerance, while others have not. Thus, we evaluated the expression of Foxp-3; a marker for Tregs, in draining lymph node homogenates from the CCR4−/− recipients. There are reductions in Foxp-3 mRNA expression by qPCR from the CCR4−/−, as compared to CCR4+/+ recipient lymph nodes at day 7 (FIGS. 2K and 2L). These results suggest the inhibition of CCR4 with its ligands can decrease Foxp-3 expressing cells in SLT during an allogeneic response. Overall, CCR4 deletion leads to diminished T and Foxp-3 expressing cells within the SLT following allograft transplantation.

As shown in FIG. 4, CCR4−/− allograft recipients have reduced T cells in draining lymph nodes at day 7. CCR4−/− as compared to CCR4+/+ allograft recipient draining lymph nodes were harvested for flow cytometry. (A-C) Representative flow cytometry plots of whole draining lymph node single cell suspensions demonstrating the gating strategy used to evaluate the frequency of CD4⁺ and CD8⁺ T cells and their naïve (Tn) and central memory (CM) cell subpopulations.

### Example 5

CCR4 Deficiency on T Cells Inhibits Allograft Rejection by Preventing Tn Homing and Activation within the Draining SLT

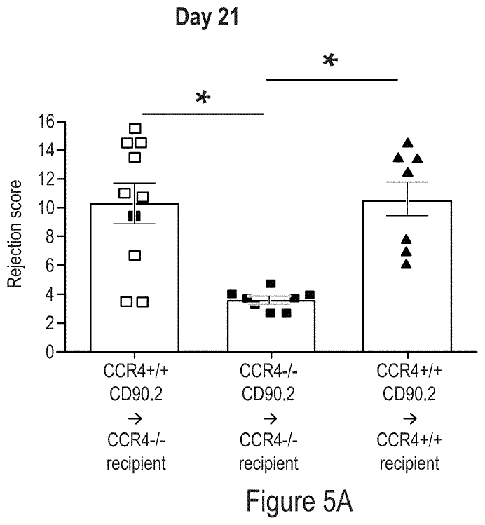

To probe whether CCR4 signaling specifically, on T cells, attenuates allograft rejection, T cells from CCR4+/+ or CCR4−/− mice were adoptively transferred pre-transplant (day 0) into CCR4+/+ or CCR4−/− recipients. Airway allografts in CCR4−/− recipients receiving CCR4−/− T cells had limited rejection, while airway allografts in CCR4−/− recipients receiving CCR4+/+T cells were rejected similar to CCR4+/+ allograft recipients with the adoptive transfer (AT) of CCR4+/+ T cells (FIG. 5A). Analysis of the airway allograft tissue demonstrate that the CCR4−/− recipients with the AT of CCR4−/− T cells have some intraluminal leukocyte infiltration as well as mucus with cytokeratin and leukocyte debris that is inherent to the heterotopic position of the airway graft (FIG. 5B). Importantly, there is minimal epithelial cell injury without any significant fibroblasts obstructing the airway (FIG. 5B). In opposition, CCR4−/− recipients with the AT of CCR4+/+ T cells are similar to the CCR4+/+ recipients receiving CCR4+/+ T cells, in which we observe many intraluminal leukocytes and a denuded basement membrane with fibroblasts obstructing the airway (FIG. 5B). Thus, CCR4 expression on the T cells is critical for driving allograft rejection, while preventing CCR4-ligand interactions dramatically attenuates graft rejection.

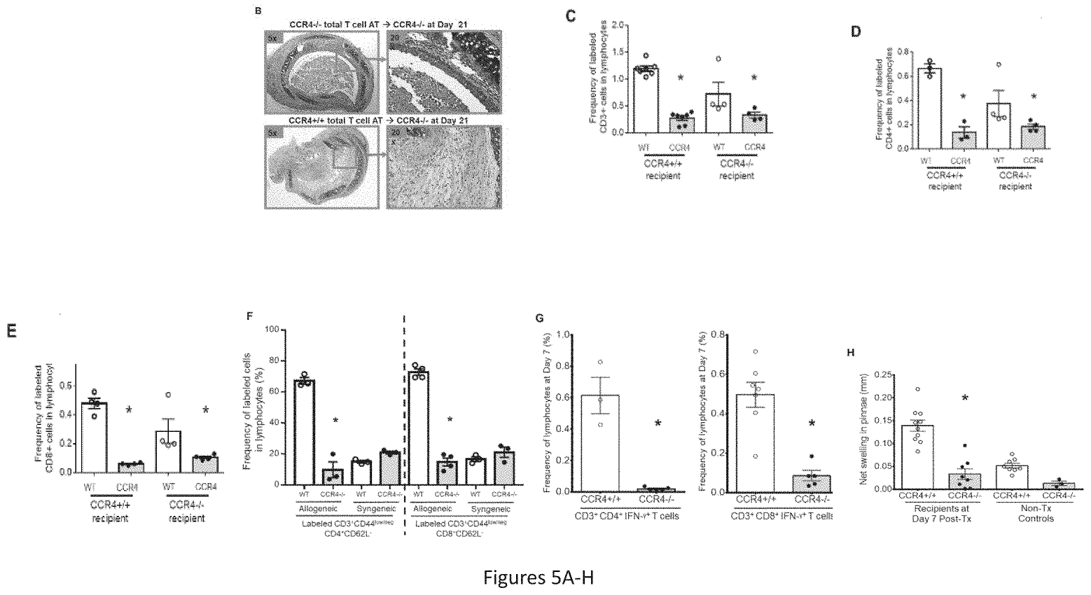

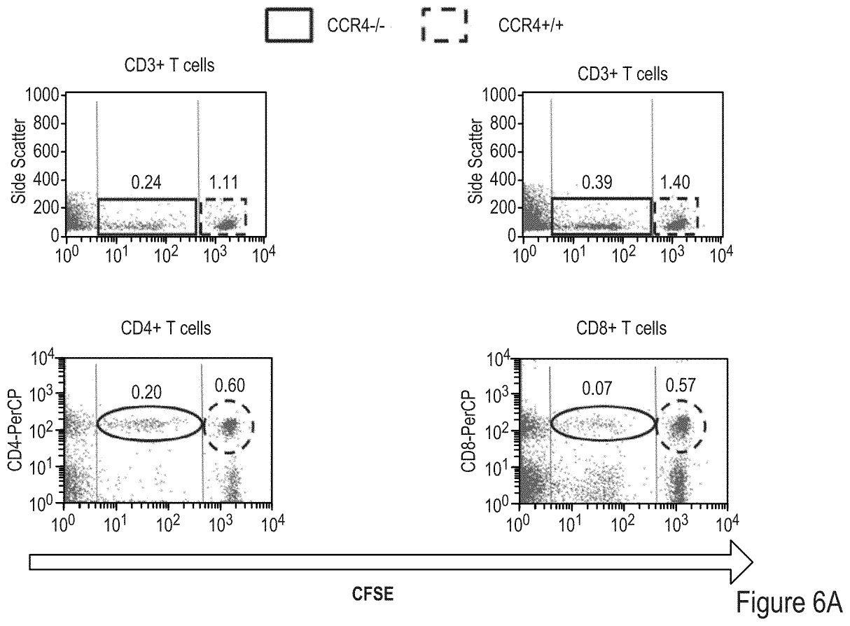

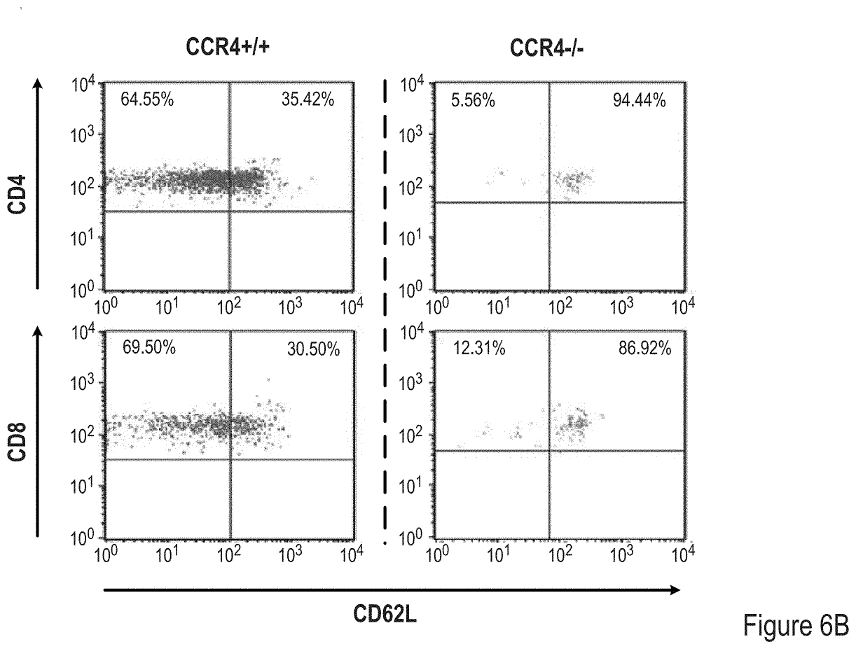

Exploring mechanisms for CCR4 expressing T cells involvement in rejection, CCR4+/+ and CCR4−/− T cells from naïve mice were labeled and equal amounts transferred into day 7 CCR4+/+ recipients of airway allografts. Eighteen hours post-transfer, the draining lymph nodes were harvested and processed into single cell suspensions for labeled T cell analysis by flow cytometry. There were dramatic reductions in the frequency of labeled CCR4−/− total T cells as well as CD4 and CD8 T cells in allograft draining nodes, as compared to labeled CCR4+/+ T cells and their subpopulations (FIGS. 5C-5E, and FIG. 6A). However, if there are certain genetic differences between the CCR4−/− and CCR4+/+ T cells, it is possible that the endogenous and transferred CCR4+/+ T cells might reject co-transferred CCR4−/− T cells and this would result in a lower recovery of the CCR4−/− T cells within the draining nodes. Thus, we performed the same co-transfer experiments using day 7 CCR4−/− allograft recipients. Again, there were reductions in the frequency of labeled CCR4−/− total T cells, CD4 and CD8 T cells in the CCR4−/− allograft recipient draining nodes, as compared to labeled CCR4+/+ T cells and their subpopulations (FIG. 5C-5E). However, not to the same magnitude found in the day 7 CCR4+/+ recipients, likely due to less rejection found with the CCR4−/− recipients. Furthermore, we found CD62L was downregulated on the majority of labeled CCR4+/+CD4⁺ and CD8⁺ Tn cells in the SLT from the CCR4+/+ allograft recipients at 18 hours, while CD62L expression remained high on almost all of the labeled CCR4−/− Tn cell CD4⁺ and CD8+ subpopulations (FIG. 5F, and FIG. 6B). There is a possibility that the downregulation of CD62L by CCR4+/+ T cells is due to their activation by the CCR4−/− co-transferred T cells if they have certain genetic differences from the CCR4+/+ T cells rather than being activated from alloantigens. Hence, we performed the same co-transfer experiment, but into day 7 isografts and found there was no differences in CD62L expression, which remained high on most of the CCR4+/+ and CCR4−/− labeled Tn cell subpopulations (FIG. 5F), confirming that CCR4−/− as compared to CCR4+/+ T cells are having trouble being activated from alloantigens within the draining lymph node. Collectively, these experiments indicate that the CCR4−/− CD4⁺ and CD8⁺ Tn cells during rejection have decreased ability to home to draining lymph nodes and those CCR4−/− T cells that do make it to the lymph nodes are not being efficiently activated against alloantigens from the airway allograft.

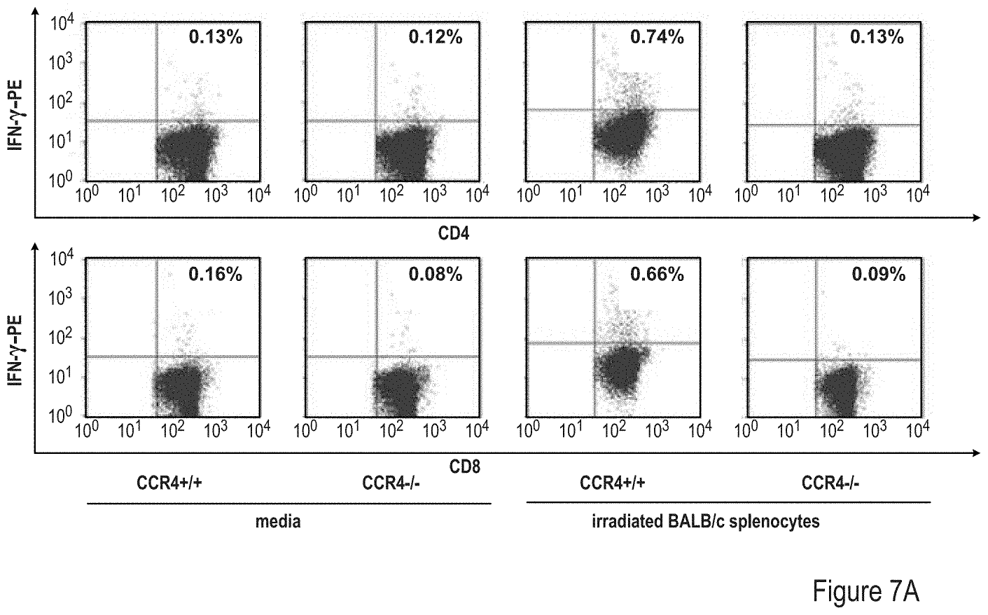

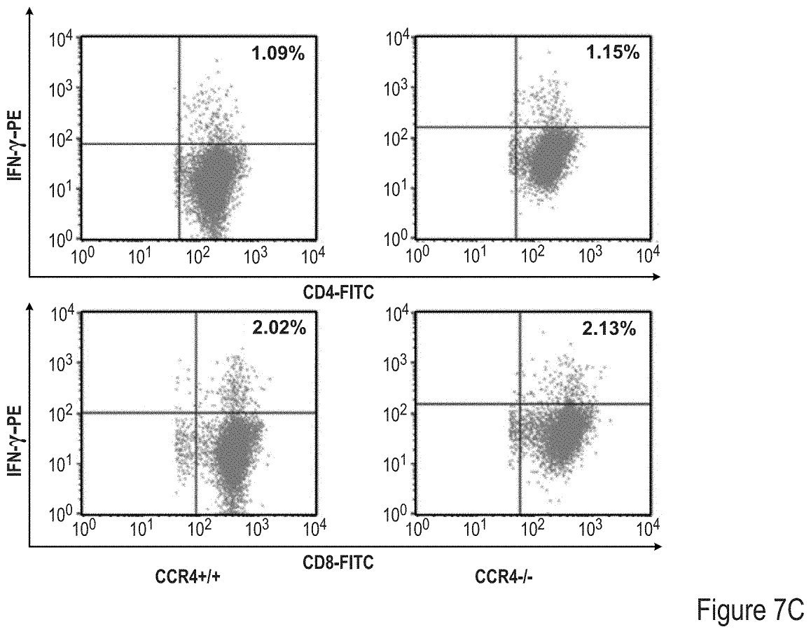

As shown in FIG. 5, CCR4 expression is involved in naïve T cell (Tn) homing and intranodal activation. CD90.2 (1×10⁷) T cells from either CCR4−/− or CCR4+/+naïve mice were transferred to either CCR4−/− or CCR4+/+ recipients on day 0 and the allografts were analyzed for rejection scores at day 21. (A) Bar graph indicates the rejection scores. (B) Representative H&E staining showing that the transfer of CCR4+/+ T cells to CCR4−/− recipients leads to severe rejection with a denuded epithelium and fibro-obliteration. However, the transfer of CCR4−/− T cells to CCR4−/− recipients has no significant epithelial injury or fibro-obliteration. In separate experiments, CFSE labeled T cells from CCR4+/+(4.0 uM, 5×10⁶) and CCR4−/− (0.25 uM, 5×10⁶) naïve mice were transferred at a 1:1 ratio into day 7 CCR4+/+ and CCR4−/− allograft recipients or isograft (C57BL/6 airways to C57BL/6 recipients), and 18-hours later the draining nodes were analyzed for the frequency of labeled T cells as well as their activation based on CD62L shedding using flow cytometry. (C-E) Bar graphs depict the frequency of labeled CCR4−/− and CCR4+/+CD3⁺, CD4⁺ and CD8⁺ T cells. See also FIG. 6. (F) Bar graph depicts the frequency of labeled CD4⁺ and CD8⁺ T cells from CCR4−/− and CCR4+/+naïve mice that entered the lymph nodes and lose CD62L expression. See also FIG. 6. (G) Day 7 CCR4−/− and CCR4+/+ allograft recipients draining lymph node cells (1×10⁶) were challenged with (2×10⁶) irradiated-BALB/c splenocytes and 16-hours later analyzed for alloresponsive CD4⁺ and CD8⁺ T cells via IFN-g secretion. See also FIG. 7. (H) Day 7 CCR4−/− and CCR4+/+ allograft recipients were challenges with (7.5×10⁶) irradiated-BALB/c splenocytes with an intra-dermal injection into the pinna and analyzed for DTH response at 48-hours. Data is representative of 3-12 mice per group. Error bars indicate SEM. Significance was determined by Mann-Whitney, unpaired t test, or Kruskal-Wallis with post-hoc Dunn where appropriate, *p<0.05.

As shown in FIG. 6, CCR4 expression on T cells is important for homing to draining lymph nodes as well as intranodal T cell activation. CCR4+/+ and CCR4−/− T cells were labeled with (4.00 μM) and (0.25 μM) of CFSE; respectively, mixed 1:1 and delivered to day 7 CCR4+/+ allograft recipients. Eighteen hours later the draining nodes were prepared for flow cytometry. (A) Representative flow cytometry plots of whole lymph node single cell suspensions demonstrating the gating strategy used to evaluate the frequency of labeled CD3⁺, CD3⁺CD4⁺, and CD3⁺CD8⁺ T cells after transfer from naïve CCR4−/− versus CCR4+/+ mice. (B) Representative flow cytometry plots of whole lymph node single cell suspensions demonstrating the gating strategy used to evaluate naïve T cell (Tn) shedding of the adhesion molecule, CD62L via gating on the CCR4−/− versus CCR4+/+CD4⁺CFSE⁺CD3⁺CD44^{low/neg} and CD8⁺CFSE⁺CD3⁺CD44^{low/neg} subpopulations.

### Example 6

CCR4−/− Recipients have a Reduction in Alloresponsive CD4⁺ and CD8⁺ T Cells

The reduction in SLT homing and intranodal activation of CD4⁺ and CD8⁺ Tn cells following allograft transplantation, suggests that CCR4−/− T cells would also exhibit decreased effector function. To test this assumption cells were isolated from the draining SLT 7 days after allograft transplantation from CCR4−/− and CCR4+/+ recipients and stimulated with irradiated BALB/c splenocytes. In response to allo-stimulation, CCR4+/+CD4⁺ and CD8⁺ T cells readily produced high amounts of IFN-γ (FIG. 5G, and FIG. 7A). Strikingly, CD4⁺ and CD8⁺ T cells from CCR4−/− mice produced almost no IFN-γ in response to allo-stimulation despite the graft having been transplanted 7 days earlier (FIG. 5G, and FIG. 7A). Importantly, both CCR4−/− and CCR4+/+ SLT CD4⁺ and CD8⁺ T cells from naïve mice exhibited similar responses to the superantigen Staphylococcal Enterotoxin B with regard to IFN-γ production, demonstrating that CCR4−/− T cells do not have an intrinsic activation defect (FIGS. 7B and 7C).

To further test the induction of the allograft response, we used an in vivo delayed type hypersensitivity (DTH) response to alloantigens as a physiologic readout of alloprimed cells. Irradiated BALB/c splenocytes were administered intradermally to the pinnae of: (1) CCR4−/− allograft recipients at day 7 after transplant; (2) CCR4+/+ allograft recipients at day 7 after transplant; (3) naive CCR4−/−; or (4) naïve CCR4+/+ mice. CCR4−/− allograft recipients displayed a markedly reduced DTH response compared to CCR4+/+ allograft recipients (FIG. 5H). Furthermore, the response was reduced to the level of that seen in the naive CCR4−/− and naïve CCR4+/+ mice (FIG. 5H), indicating minimal functionally active alloresponsive T cells in the absence of CCR4 expression.

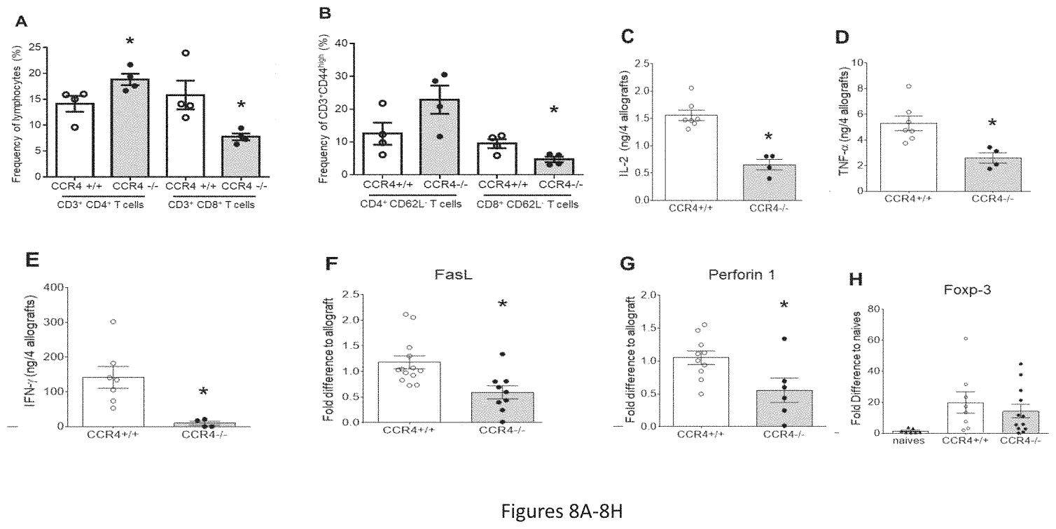

We further explored the down-stream effects of CCR4-ligand inhibition on T cell function within the airway allograft. Interestingly, the frequency of allograft infiltrating CD4+ T cells in CCR4−/− mice was significantly increased, whereas the frequency of CCR4−/− CD8+T cells was markedly decreased compared to CCR4+/+ mice 7 days after allograft transplantation (FIG. 8A). Upon further analysis there was no difference in CD44^{high} CD4+CD62L-T cells and a reduction in the frequency of CD44^{high} CD8⁺CD62L⁻ T cells in the allografts from the CCR4−/− recipients as compared to the CCR4+/+ recipients (FIG. 8B). Furthermore, the protein levels of IL-2, TNF-α and IFN-γ, and the mRNA levels of the T cell killing effectors FasL and perforin were also decreased in the airway allografts of CCR4−/− mice as compared to the CCR4+/+ recipients (FIGS. 8C-8G). In contrast, there was no significant difference in the mRNA expression of Foxp-3 in airway allografts from CCR4−/− and CCR4+/+ recipients (FIG. 8H) Thus, in the absence of CCR4 expression, there is a dramatically diminished ability for T cells to mount functional responses against airway allografts, despite being present in the transplanted tissue.

As shown in FIG. 7, CCR4−/− allograft recipients have a reduction in the clonal expansion of CD4⁺ and CD8⁺ T cells. Day 7 CCR4−/− versus CCR4+/+ airway allograft recipient's draining lymph nodes single cell suspensions were challenged ex vivo for 16 hours with donor irradiated BALB/c splenocytes and analyzed for T cell secretion of IFN-g via flow cytometry. In separate experiments CCR4−/− versus CCR4+/+ T cells from naïve mice were challenged with superantigen Staphylococcal Enterotoxin B for 16 hours to determine T cell secretion of IFN-g. (A) Representative example of the flow cytometry gating strategy evaluating recipient draining nodes alloresponsive CD4⁺ and CD8⁺ T cells secreting IFN-γ via gated on CD3⁺CD4⁺IFN-γ+ and CD3⁺CD8⁺IFN-γ+ cells. (B) Bar graph depicts CD4⁺ and CD8+ T cells from naïve CCR4−/− and CCR4+/+ mice that express IFN-g after superantigen Staphylococcal Enterotoxin B challenge. (C) Representative example of flow cytometry gating strategy evaluating CD4⁺ and CD8⁺ T cells secreting IFN-γ from naïve mice after superantigen Staphylococcal Enterotoxin B challenge via gated on CD3⁺CD4⁺IFN-γ+ and CD3⁺CD8⁺IFN-γ+ cells. Data is representative of 3-12 mice per group. Error bars indicate SEM. Significance was determined by Mann-Whitney, *p<0.05.

As shown in FIG. 8, CCR4−/− recipients have a reduction in airway allograft infiltrating cytotoxic lymphocytes and their mediators. Day 7 whole airway allografts from CCR4−/− and CCR4+/+ recipients were analyzed for allograft infiltrating T cell subpopulations and cytotoxic mediators using flow cytometry, luminex and qPCR. (A-B) Bar graphs depict the frequency of allograft infiltrating, CD4⁺ and CD8⁺ T cells as well as their subpopulations. (C-E) Bar graphs depict the whole allograft homogenates protein concentrations for IL-2, TNF-α, and IFN-γ. (F-H) Bar graphs represent the whole allograft homogenates mRNA expression for FasL, perforin-1 and Foxp-3. Data is representative of 4-7 mice per group. Error bars indicate SEM. Significance was determined by Mann-Whitney, *p<0.05.

### Example 7

Allograft Rejection is Dependent on Help from CCR4+/+CD4⁺ T Cells, not NKT or CCR4+/+CD8⁺ T Cells

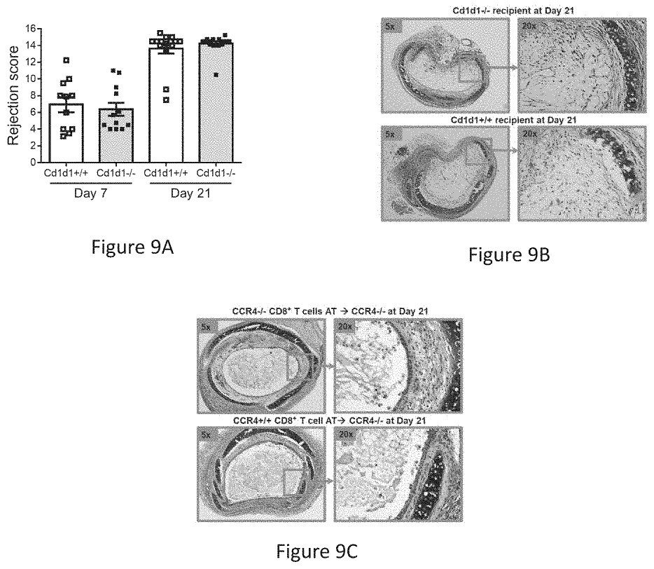

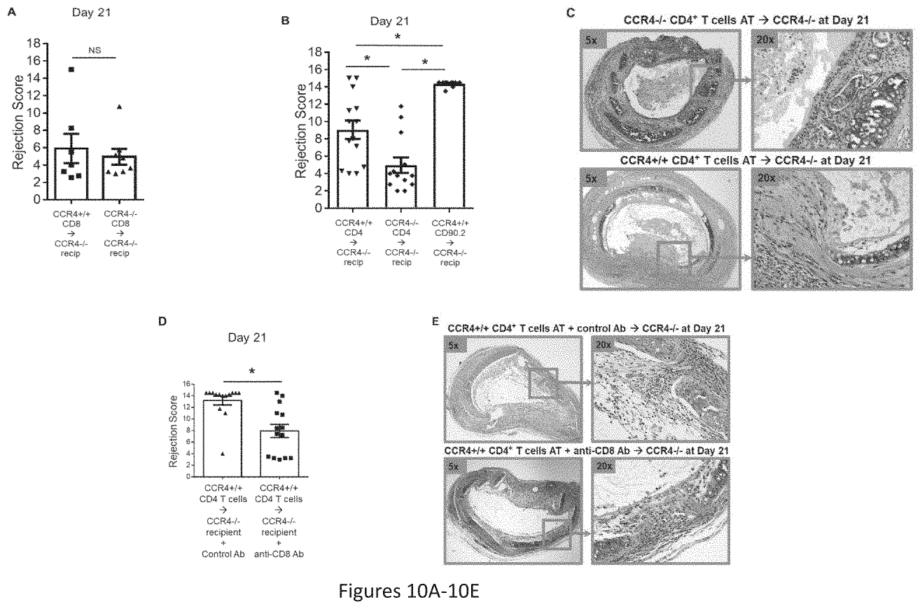

We observed no difference in airway rejection scores at days 7 and 21 between Cd1d1−/− recipients that lack NKT cells as compared to Cd1d1+/+ recipients (FIGS. 9A and 9B). To determine if other subsets of T cells functioned in a CCR4 dependent mechanism to induce allograft rejection, we adoptively transferred CCR4+/+CD4⁺ T cells, CD8⁺ T cells, or total T cells into CCR4−/− recipients just prior to airway transplantation. At day 21 post-transplant, the CCR4−/− recipients receiving either CCR4+/+ or CCR4−/− CD8⁺ T cells exhibited a similarly low rejection score (FIG. 10A, and FIG. 9C). Interestingly, CCR4−/− recipients receiving CCR4+/+CD4⁺ T cells re-established a significant portion of alloreactivity with rejection scores markedly greater than transferred CCR4−/− CD4⁺ T cells, but not as high as the scores found with the transfer of total T cells to the CCR4−/− recipients (FIGS. 10B and 10C). Furthermore, when CCR4+/+CD4⁺ T cells were transferred to CCR4−/− recipients with either control Ab or the depleting anti-CD8 Ab delivered at days −1, 7 and 14, there was a reduction in rejection scores with the anti-CD8 Ab treated group at day 21 (FIGS. 10D and 10E). Collectively, these results imply that maximal allograft rejection is present when CCR4 expression is on both CD4⁺ and CD8⁺ T cells and suggest that CCR4 expressing CD4⁺ T cells can directly cause a degree of allograft injury as well as provide help to CD8⁺ T cells during allograft rejection.

As shown in FIG. 9, Cd1d−/− recipients as well as the adoptive transfer of CCR4+/+CD8⁺ T cells to CCR4−/− recipients does not affect airway rejection. BALB/c airways were transplanted into C57BL/6 Cd1d−/− versus Cd1d+/+ mice. In separate experiments (5×10⁶) CD8⁺ T cells from either CCR4−/− or CCR4+/+naïve mice were transferred to CCR4−/− recipients of BALB/c airway grafts on day 0 and the allografts were analyzed for rejection scores at day 21. (A) Bar graph depicts the rejection scores of airway allografts from Cd1d−/− as compared to Cd1d+/+ recipients at days 7 and 21. (B) Representative hematoxylin and eosin (H&E) staining of transplanted BALB/c airway allografts from Cd1d−/− and Cd1d1+/+ recipients at day 21, both demonstrating intraluminal inflammation, loss of the epithelium and fibro-obliteration. (C) Representative H&E staining of allograft airways from CCR4−/− recipients after day 0 adoptive transfer of CD8⁺ T cells from either CCR4+/+ or CCR4−/− naïve mice both demonstrating mild intraluminal inflammation, preserved epithelium and no fibro-obliteration at day 21. Data is representative of 10-16 mice per group. Error bars indicate SEM. Significance was determined by Mann-Whitney, *p<0.05.

As shown in FIG. 10, the hierarchy for T cells re-establishing rejection in the CCR4−/− recipient mice. CD8⁺ (5×10⁶), CD4⁺ (5×10⁶) and CD90.2⁺ (1×10⁷) T cells from either CCR4−/− or CCR4+/+naïve mice were transferred to CCR4−/− recipients of BALB/c airway grafts on day 0 and the allografts were analyzed for rejection scores at day 21. Bar graph indicates the rejection scores of allografts from CCR4−/− recipients with the transfer of (A) CD8 T cells and (B) CD4 and CD90.2 T cells. See also FIG. 9. (C) Representative H&E staining of day 21 transplanted BALB/c airways from adoptively transferred CD4⁺ T cells from either CCR4+/+ or CCR4−/− naïve mice transferred into CCR4−/− recipients at day 0. CCR4−/− recipients with transferred CCR4−/− CD4⁺ T cells have a virtually normal epithelium without fibroblasts obstructing the lumen. Allografts from the CCR4−/− recipients with transferred CCR4+/+CD4+ T cells have a denuded airway epithelium with a moderate amount of fibroblasts invading the airway lumen. In separate experiments, CCR4+/+CD4⁺ T cells (5×10⁶) were transferred to CCR4−/− recipients with either control Ab or the depleting anti-CD8 Ab delivered at days −1, 7 and 14, and the allografts were analyzed for rejection scores at day 21. (D) Bar graph depicts the rejection scores of allografts from CCR4−/− recipients with the transfer of CCR4+/+CD4+ T cells with or without anti-CD8 Ab. (E) CCR4−/− recipients with transferred CCR4+/+CD4+ T cells plus a control Ab have a loss of airway epithelium with a moderate amount of fibroblasts obstructing the lumen. CCR4−/− recipients with transferred CCR4+/+CD4⁺ T cells plus an anti-CD8 Ab have a denuded airway epithelium and minimal airway invading fibroblasts. Data is representative of 10-16 mice per group. Error bars indicate SEM. Significance was determined by Mann-Whitney or Kruskal-Wallis with post-hoc Dunn where appropriate. *p<0.05.

### Example 8

CCR4 Deletion in Combination with CTLA4-Ig Leads to Long-Term Airway and Lung Allograft Accommodation

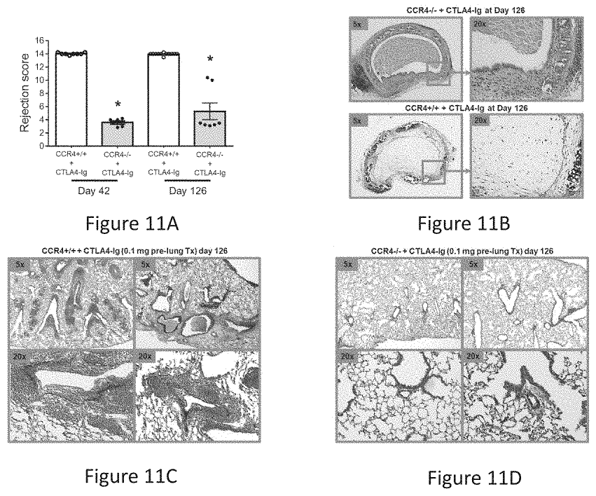

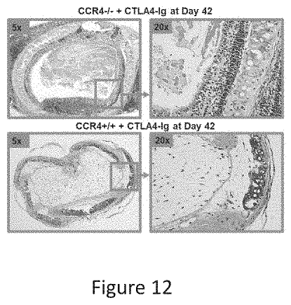

The combination of anti-CD154 (CD40L) with CTLA4-Ig to inhibit T cell priming is considered the preeminent combination used to induce long-term allograft accommodation. Therefore, we hypothesized that CCR4-ligand inhibition by decreasing T cell priming and activation may have a similar therapeutic potential as anti-CD154 to enhance CTLA4-Ig immunotherapy. Accordingly, we evaluated the effects of CCR4−/− allograft recipients receiving CTLA4-Ig therapy on allograft survival. CTLA4-Ig (0.2 mg) i.p. was given on day 0 prior to transplant and at days 2, 4 and 6 post-transplant to CCR4−/− or CCR4+/+ recipients of BALB/c airways. Strikingly, the allograft airways from the CCR4−/− recipients are virtually normal at days 42 and 126; whereas the allograft airways from the CCR4+/+ recipients were completely rejected and fibro-obliterated by day 42 (FIGS. 11A and 11B, FIG. 12). Thus, CCR4 deficiency in combination with a short peri-operative course of CTLA4-Ig enabled an unprecedented long-term allograft survival in a situation that normally leads to rejection in a month.

We next performed proof of concept studies using the fully mismatched, vascularized left single lung transplant model via the same stringent strain combination (BALB/c lung to C57BL/6 recipients) as the airway allograft model to corroborate our results. Lung allografts in CCR4+/+ recipient mice treated with CTLA4-Ig (0.1 mg) i.p. just prior to transplant developed severe grade A rejection (AR≥3) with or without vasculitis, severe grade B rejection (B≥3) with high-grade infiltrates and epithelial cell injury, and severe pleural and septal inflammation with and without fibrosis by day 126 post-transplant (FIG. 11C). In contrast, CCR4−/− recipients treated with the same single dose of pre-transplant CTLA4-Ig (0.1 mg) had virtually normal lung allografts without any significant pathology at day 126 (FIG. 11D). Quantitatively, CCR4+/+ versus CCR4−/− recipients with a single pre-transplant low dose of CTLA4-Ig, allograft scores are (AR: 100% vs 0%; p=0.008), (LB: 100% vs 0%; p=0.008), (pleural involvement: 60% vs 0%; p=0.17), (septal involvement: 80% vs 0%; p=0.04); respectively. Thus, CCR4 deficiency in combination with an initial CTLA4-Ig treatment at the time of transplantation enabled long-term lung allograft accommodation.

As shown in FIG. 11, CTLA4-Ig combined with CCR4−/− recipients leads to long-term allograft accommodation in two models of allograft rejection. CTLA4-Ig given intraperitoneal (i.p.) at 0.2 mg on day 0 prior to airway transplantation and at days 2, 4, and 6 post-transplant to CCR4−/−, as compared to CCR4+/+ recipients were evaluated for rejection. (A) Bar graph indicates the rejection scores for CTLA4-Ig treated CCR4−/− and CCR4+/+ allograft recipients at days 42 and 126. See also FIG. 12. (B) Day 126 representative H&E staining of airway allografts from CCR4−/− and CCR4+/+ recipients given CTLA4-Ig. CTLA4-Ig given to CCR4−/− recipients demonstrate virtually normal airways with intact ciliated epithelium and no fibro-obliteration. CTLA4-Ig given to CCR4+/+ recipients leads to airways that are rejected, without epithelium, and invaded by fibroblasts causing fibro-obliteration. Section of airway allograft is magnified to show the presence of the epithelial layer in CTLA4-Ig+CCR4−/− and absence of the epithelial layer with fibro-obliteration in the CTLA4-Ig+CCR4+/+ recipients. Orthotopic single lung transplant to CCR4−/− and CCR4+/+ recipients given CTLA4-Ig i.p. (0.1 mg) at day 0 just prior to transplant. (C) Day 126 representative elastin trichrome stain shows that CTLA4-Ig given to CCR4+/+ allograft recipients leads to high grade vascular, airway and pleural rejection with fibrosis. (D) Day 126 representative elastin trichrome stain shows that CTLA4-Ig given to CCR4−/− allograft recipients leads to virtually normal lung allografts. Data is representative of 4-12 mice per group. Error bars indicate SEM. Significance was determined by Mann-Whitney, *p<0.05.

As shown in FIG. 12, CTLA4-Ig combined with CCR4−/− recipients leads to long-term airway allograft accommodation. CTLA4-Ig given intraperitoneal (i.p.) at 0.2 mg on day 0 prior to airway transplantation and at days 2, 4, and 6 post-transplant to CCR4−/− versus CCR4+/+ recipients and grafts harvested to quantitate rejection at day 42. (A) Representative H&E staining of CCR4−/− and CCR4+/+ recipients given CTLA4-Ig at day 42. CTLA4-Ig given to CCR4−/− recipients demonstrate virtually normal airways with intact ciliated epithelium and no fibro-obliteration. CTLA4-Ig given to CCR4+/+ recipients leads to airways that are rejected and invaded by fibroblasts causing fibro-obliteration. Section of airway allograft is magnified to show the presence of the epithelial layer in CTLA4-Ig+CCR4−/− and absence of the epithelial layer with fibro-obliteration in the CTLA4-Ig+CCR4+/+ recipients.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for treating or preventing organ injury, delayed type hypersensitivity, graft-versus-host disease or allograft rejection in a subject comprising administering to the subject an effective amount of a CCR4 chemokine receptor inhibitor or antagonist.

2. The method of claim 1 wherein the organ injury is lung injury.

3. The method of claim 2 wherein lung injury is selected from acute lung injury, pulmonary fibrosis and sequelae of allogeneic lung transplantation.

4. The method of claim 1 wherein the allograft rejection is of a kidney, heart, liver, lung, extremity, face or pancreas.

5. (canceled)

6. The method of claim 1, wherein the CCR4 inhibitor or antagonist is a small molecule or an antibody.

7. The method of claim 6, wherein the antibody is mogalizumab, mogalizumab-kpkc, or KH-4F5.

8. The method of claim 6 wherein the small molecule is CCX6239, FLX475, BMS-397, AZD-2098, AZD-1678, GSK2239633, 1′-{4-[(4-chlorophenyl)amino]-6,7-dimethoxyquinazolin-2-yl}-1,4′-bipiperidin-3-yl)methanol, 4-[4-chloro-6-(2,4-dichloro-benzylamino)-pyrimidin-2-yl]-piperazin-1-yl}-piperidin-2-yl-methanone, C-021 dihydrochloride or RPT193.

9. The method of claim 1, further comprising administering to the subject a CTLA4 inhibitor.

10. The method of claim 9, wherein the CTLA4 inhibitor is abatacept or ipilimumab.

11. The method of claim 4 further comprising administering to the subject one or more compounds selected from the group consisting of cyclosporin, tacrolimus (FK506), sirolimus (rapamycin), methotrexate, mycophenolic acid (mycophenolate mofetil), everolimus, daphnoretin, dexamethasone, prednisone, azathioprine, fluorouracil, mercaptopurine, anthracycline, bleomycin, dactinomycin, mithramycin, mitomycin, and NOX-100.

12. The method of claim 6, wherein the CCR4 inhibitor or antagonist is administered in an amount comprising from about 0.5 to about 50 mg/kg/day.

13. The method of claim 6, wherein the CCR4 inhibitor or antagonist is administered in an amount comprising from about 1 to about 5 mg/kg/day.

14. The method of claim 6, wherein the CCR4 inhibitor or antagonist is administered orally to the subject.

15. The method of claim 6, wherein the CCR4 inhibitor or antagonist is administered parenterally to the subject.

16. The method of claim 6, wherein the CCR4 inhibitor or antagonist is administered for between about 1 to about 180 days.

17. The method of claim 6, wherein the CCR4 inhibitor or antagonist is administered for an indefinite period of time to maintain inhibition of transplant rejection.

18. The method of claim 6, wherein the CCR4 inhibitor or antagonist is administered to the subject prior to a tissue or organ transplant.

19. (canceled)

20. The method of claim 6, further comprising exposing the CCR4 inhibitor or antagonist to the tissue or organ ex vivo prior to transplantation into the subject.

21.-37. (canceled)
